(19) United States
(12) Patent Application Publication
(10) Pub. No.: US 2015/0080926 A1
(43) Pub. Date: Mar. 19, 2015

(54) ULTRASOUND APPARATUSES, SYSTEMS, AND METHODS FOR RENAL NEUROMODULATION

(71) Applicant: Medtronic Ardian Luxembourg S.a.r.l., Luxembourg (LU)

(72) Inventor: Charles Emery, Sammamish, WA (US)

(73) Assignee: Medtronic Ardian Luxembourg S.a.r.l., Luxembourg (LU)

(21) Appl. No.: 14/397,452

(22) PCT Filed: Feb. 25, 2013
(86) PCT No.: PCT/US13/27593

§ 371 Date: Oct. 27, 2014

Related U.S. Application Data
(60) Provisional application No. 61/639,771, filed on Apr. 27, 2012.

(51) Int. Cl. A61N 7/00; A61B 19/00

(52) U.S. Cl. CPC A61N 7/00; A61B 19/5225; A61N 2007/003; A61B 2019/5276; A61B 2019/5236; A61B 2019/524
USPC 606/169

(57) ABSTRACT

Medical devices, systems, and methods for achieving renal neuromodulation by extracorporeal application of energy are disclosed herein. One aspect of the present disclosure is directed to apparatuses, systems, and methods that incorporate a device that employs high-intensity focused ultrasound. The high-intensity focused ultrasound may be used for application of energy to modulate neural fibers that contribute to renal function, or to vascular structures that feed or perfuse the neural fibers. The ultrasound transducer for delivering the energy may be located remotely from the desired treatment area and/or may be located outside the patient's body. In particular embodiments, an ultrasound transducer may be coupled to a targeting system that may be extracorporeal or intravascular.

### CROSS REFERENCE TO RELATED APPLICATION(S)

The present application claims the benefit of and priority to U.S. Provisional Patent Application No. 61/639,771, filed Apr. 27, 2012, which is incorporated herein by reference in its entirety.

### TECHNICAL FIELD

The present disclosure relates to ultrasound apparatuses, systems, and methods for neuromodulation. In particular, several embodiments relate to high-intensity focused ultrasound apparatuses for the application of energy to nerves proximate a renal artery.

### BACKGROUND

Hypertension, heart failure, chronic kidney disease, insulin resistance, diabetes, and metabolic syndrome represent significant and growing global health issues. Current therapies for these conditions include non-pharmacological, pharmacological, and device-based approaches. Despite this variety of treatment options, the rates of control of blood pressure and the therapeutic efforts to prevent the progression of these disease states and their sequelae remain unsatisfactory. Although the reasons for this situation are manifold and include issues of non-compliance with prescribed therapy, heterogeneity in responses both in terms of efficacy, adverse event profile, and others, it is evident that alternative options would be useful to supplement the current therapeutic treatment regimes for these conditions.

### BRIEF DESCRIPTION OF THE DRAWINGS

FIG. 1 is a conceptual illustration of the sympathetic nervous system (SNS) and how the brain communicates with the body via the SNS.

FIG. 2 is an enlarged anatomic view of nerves innervating a left kidney to form a renal plexus around the left renal artery.

FIGS. 3A and 3B are, respectively, anatomic and conceptual views of a human body depicting neural efferent and afferent communication between the brain and kidneys.

FIGS. 4A and 4B are, respectively, anatomic views of the arterial and venous vasculatures of a human.

FIG. 5 is a view of a system for high-intensity focused ultrasound renal neuromodulation including an external ultrasound energy generator and an external treatment device.

FIG. 6 is a view of a system for high-intensity focused ultrasound renal neuromodulation including an external ultrasound energy generator and an external treatment device as well as an intravascular targeting system that is inserted within a patient's vascular system.

FIG. 7 is a view of a treatment system that includes a magnetic receiver associated with a targeting element on an intravascular device positioned within or near a renal artery.

FIG. 8 is a view of a treatment system that includes a magnetic transmitter associated with a targeting element on an intravascular catheter positioned within or near a renal artery.

FIG. 9 is a view of a treatment system that includes an external acoustic transmitter as well as an acoustic receiver associated with a treatment device and with a targeting element including an acoustic receiver on an intravascular device positioned within or near a renal artery.

FIG. 10 is a view of a treatment system that includes an acoustic transmitter and receiver associated with a targeting element on an intravascular device.

FIG. 11 is a view of a treatment system that includes a separate acoustic transmitter associated with a targeting element on an intravascular device.

FIG. 12 is a view of a treatment system that includes an actuatable acoustic transmitter associated with a targeting element on an intravascular device.

FIG. 13 is a view of a treatment system that includes an acoustic receiver associated with a targeting element on an intravascular device.

FIG. 14 is a view of a treatment system that includes an annular transducer.

FIG. 15 is a view of a treatment system that includes multiple acoustic receivers associated with a targeting element on an intravascular device.

FIG. 16 is a view of a treatment system that includes a transceiver associated with a targeting element on an intravascular device.

FIG. 17 is a view of a treatment system that includes a targeting material associated with a targeting element on an intravascular device.

FIG. 18 is a view of a treatment device that includes an actuatable transducer.

FIG. 19 is a view of a treatment device that includes a patient water belt around which the therapy device is configured to move.

### DETAILED DESCRIPTION

The present disclosure is directed to apparatus, systems, and methods for achieving ultrasound-induced renal neuromodulation. Ultrasound-induced renal neuromodulation can include, for example, the partial or complete incapacitation or other effective disruption of nerves innervating the kidneys. In particular, renal neuromodulation comprises inhibiting, reducing, and/or blocking neural communication along neural fibers (i.e., efferent and/or afferent nerve fibers) innervating the kidneys. Such incapacitation can be long-term (e.g., permanent or for periods of months, years, or decades) or short-term (e.g., for periods of minutes, hours, days, or weeks). Renal neuromodulation is expected to efficaciously treat several clinical conditions characterized by increased overall sympathetic activity, and in particular conditions associated with central sympathetic overstimulation such as hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, and sudden death. The reduction of afferent neural signals from the kidneys contributes to the systemic reduction of sympathetic tone/drive. Renal neuromodulation is expected to be useful in treating several conditions associated with systemic sympathetic overactivity or hyperactivity and can potentially benefit a variety of organs and bodily structures innervated by sympathetic nerves. For example, a reduction in central sympathetic drive may reduce insulin resistance that afflicts patients with metabolic syndrome and Type II diabetics. Additionally, osteoporosis can be sympathetically activated and might benefit from the downregulation of sympathetic drive that accompanies renal neuromodulation. A more detailed description of pertinent patient anatomy and physiology is provided below.

Embodiments of the present disclosure can incorporate a treatment device having one or more ultrasound transducers. The ultrasound transducer can be configured to deliver ultrasound energy to a renal artery from an extracorporeal location relative to a patient. In particular embodiments, the ultrasound transducer may be positioned outside the body of the patient and may include an active or passive ultrasound targeting system positioned within the body of the patient. In other embodiments, the entire system (e.g., the ultrasound transducer and the targeting system) may be extracorporeal. Regardless of the arrangement of the ultrasound transducer and targeting system, the systems as provided can be capable of creating lesions by the application of ultrasound energy to renal nerves.

Although the present disclosure is detailed and exact to enable those skilled in the art to practice the disclosed technologies, the physical embodiments disclosed herein merely exemplify the various aspects of the disclosure, which may be embodied in other specific structures. Details may be changed without departing from the disclosure, which is illustrated, though not limited, by the following examples.

### I. HIGH-INTENSITY FOCUSED ULTRASOUND FOR RENAL NEUROMODULATION

In catheter systems configured for the intravascular application of energy to vascular tissue, in order to achieve a therapeutic effect, an energy-delivery element is generally placed as close to the tissue to be treated as possible. In particular, the highest energy density is generally closest to the tip of the energy-delivery element, and the effect on tissue generally decreases as distance from the tip increases. For intravascular renal neuromodulation applications, this may result in higher energy delivery to the interior of the renal artery with less energy delivered to the renal nerves, which lay primarily within the adventitia of the renal artery beyond the interior luminal wall of the renal artery. As such, achieving suitable energy delivery that modulates the nerves without overheating the renal artery can be complex.

In cardiac ablation technologies, deep scarring of heart muscle, known as transmural lesions, can be created to control arrhythmias. The goal of renal neuromodulation can differ from that of cardiac ablation in that creation of transmural lesions in the blood vessel walls is generally not desired. Nerves are generally more fragile than other tissue and can stop conducting signals when heated but not necessarily scarred. At the same time, nerves can be located some distance away from the blood vessel luminal wall where the heating instrument may be located.

Provided herein are apparatuses, methods, and systems that can incorporate high-intensity focused ultrasound (HIFU) as an energy source to therapeutically treat tissue. Such HIFU-based systems may incorporate elements that can be extracorporeal (i.e., located outside a patient's body). For example, the systems may include one or more ultrasound transducers that can be located on the patient's body or otherwise outside the patient. The ultrasound energy may include mechanical vibrations above the threshold of human hearing. Ultrasound waves may propagate through living tissue and fluids without causing harm to cells. By focusing highly energetic ultrasound waves to a well-defined volume, local heat rise (e.g., >56° C. and typically up to 80° C.) can occur and cause rapid tissue necrosis (e.g., coagulative necrosis). Steep temperature gradients can be observed between the focus and the surrounding tissue, allowing for the production of sharply demarcated lesions and reducing collateral damage. The controlled degree of heating and damage may be achieved by dosing of energy (e.g., by controlling electric power delivered to the source and/or by controlling the duration of application of energy). Pulsed ultrasound may also be used to control tissue modification and, in particular embodiments, may be used in conjunction with other energy sources (e.g., for imaging). Furthermore, frequency selection and aperture size and/or geometry may be used to control tissue modification.

Another mechanism by which HIFU can destroy tissue is called acoustic cavitation. This process is based on vibration of cellular structures causing local hyperthermia and mechanical stress by bubble formation due to rapid changes in local pressure leading to cell death. It is appreciated that for the purpose of this disclosure, necrosis of tissue may not be needed. Nerves typically are more fragile than surrounding tissue and may be effectively functionally disabled in some cases by heating to a temperature that does not cause necrosis. In particular embodiments, the heating of selected tissue with ultrasonic waves to a temperature above normal range may be referred to as “sonication.”

HIFU presents several advantages over other energy modalities for renal neuromodulation. In particular embodiments, for example, ultrasound can be capable of focusing energy on one or more focal points some distance from the source of ultrasonic waves. As opposed to energy application from a thermal or radiofrequency source that distributes energy locally at the point of application, HIFU may focus energy at a distant point with targeted focusing of the ultrasound energy. As such, in a treatment including HIFU, an energy source may be remote (e.g., not within the renal artery or even within the body) and achieve energy application and renal neuromodulation generally without disturbing tissue located proximally or distally from the intended treatment zone. Targeted energy delivery may be achieved without precise placement of a catheter device, which may allow greater operator flexibility and may provide additional benefit to patients whose anatomy may make placement of catheter within a renal artery particularly challenging.

In particular embodiments, HIFU emitters may be configured to focus energy on deep tissue zones one to three millimeters away from the arterial wall, therefore generally avoiding the intima and media of the renal artery while still modulating nerves that may be dispersed between the adventitia and over some distance from the arterial wall. Histological studies show that renal nerves form a plexus of many fibers surrounding the external wall of the renal artery. While some may be embedded in the exterior of the arterial wall, some may be located several millimeters outside. In addition, HIFU may achieve deep tissue heating that is expected to result in complete or generally complete destruction or inactivation of renal nerves. Further, because HIFU techniques may target the nerves while sparing the arterial wall, higher levels of concentrated heat may be applied to the target, thus shortening the procedure. Furthermore, a HIFU device can focus energy at multiple focal points simultaneously which may further reduce procedure time.

Embodiments provided herein can include a HIFU transducer, an energy focusing device, and an acoustic frequency generator. In particular embodiments provided herein, a HIFU transducer may be a single element or an array of elements, for example, a sonic crystal or an array of crystals. In such arrangements, focusing of acoustic energy emitted by the crystal may be achieved via a focusing lens (e.g., a lens having a concave cavity), mechanical means (e.g., one or more motors used to move the transducer in one or multiple axes), the shape of the crystal, or the use of materials of differing velocity. The actual geometry of a cavity can determine the distance from an energy application point (e.g., the location of the HIFU transducer) to the point of energy focus. The energy focus device and/or the HIFU transducer may be equipped with a temperature sensor and temperature control circuits to prevent overheating of tissue and the device itself.

As noted, the HIFU transducer may be extracorporeal, which may facilitate remote treatment of tissue (e.g., with energy applied via a transducer not in direct contact with the treated tissue) based on energy concentration of the acoustic focus. Since most tissue is thermally insulating, the heating that occurs due to the acoustic concentration typically is not quickly conducted away from the focus. The frequency chosen for HIFU can be a function of the expected attenuation, the required containment of the beam both laterally and axially, the size and shape of the ultrasound transducer, and the geometry and location of the renal artery relative to the transducer, which can dictate the treatment depths.

Ultrasound is useful to image the soft tissues of the body, and in certain embodiments, the imaging capabilities of ultrasound techniques may be used for device placement and targeting. Furthermore, ultrasound may provide information related to flow and/or elasticity that may be beneficial for targeting. In this manner, an HIFU device for neuromodulation may be used for imaging the renal artery, targeting the renal nerves, determining the optimal treatment power or dose, and/or determining when to halt a treatment. Further, the ultrasound devices as provided herein may be used in targeting, imaging, scanning, or other appropriate operations to locate an appropriate treatment site and/or to provide treatment energy, to monitor the treatment, and/or to determine the size of the effected tissues after treatment. Such operations may include the use of Doppler ultrasound (e.g., color Doppler or power Doppler), B-mode, pulsed-wave ultrasound (e.g., A-mode or M-mode), continuous-wave ultrasound, acoustic radiation force imaging, or elasticity imaging (e.g., using shear waves). Particular embodiments may utilize therapeutic ultrasound and/or diagnostic ultrasound for successful renal neuromodulation. To that end, the relevant anatomy and physiology of renal nervous control is discussed herein.

### II. PERTINENT ANATOMY AND PHYSIOLOGY

A. The Sympathetic Nervous System

The sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the SNS operates through a series of interconnected neurons. Sympathetic neurons are frequently considered part of the peripheral nervous system, although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla. Once released, norepinephrine and epinephrine bind adrenergic receptors on peripheral tissues. Binding to adrenergic receptors causes a neuronal and hormonal response. The physiologic manifestations can include pupil dilation, increased heart rate, occasional vomiting, and increased blood pressure. Increased sweating is also seen due to binding of cholinergic receptors of the sweat glands.

The SNS is responsible for up and down regulation of many homeostatic mechanisms in living organisms. Fibers from the SNS innervate tissues in almost every organ system, providing at least some regulatory function to characteristics as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the SNS and indirectly via catecholamines secreted from the adrenal medulla.

Science typically looks at the SNS as an automatic regulation system, that is, one that operates without the intervention of conscious thought. Some evolutionary theorists suggest that the SNS operated in early organisms to maintain survival as the SNS is responsible for priming the body for action. One example of this priming is in the moments before waking, in which sympathetic outflow spontaneously increases in preparation for action.

1. The Sympathetic Chain

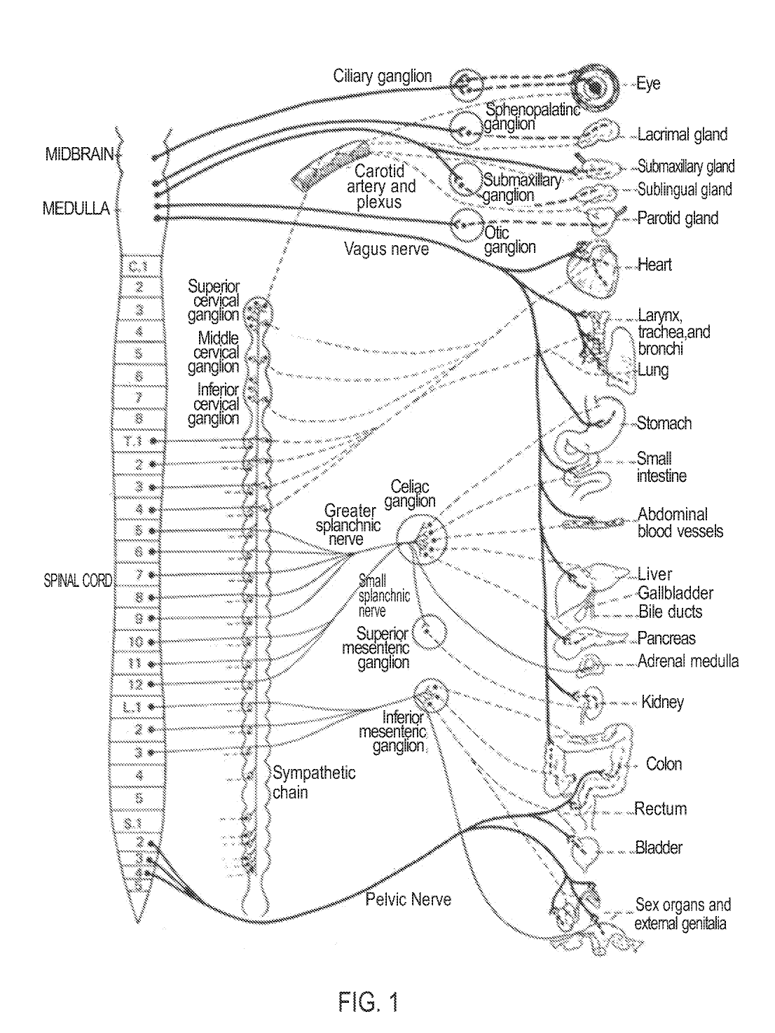

As shown in FIG. 1, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors that connect to either the paravertebral (which lie near the vertebral column) or prevertebral (which lie near the aortic bifurcation) ganglia extending alongside the spinal column.

In order to reach the target organs and glands, signals travel long distances in the body. To accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination. In the SNS and other components of the peripheral nervous system, these synapses are made at sites called ganglia. The cell that sends its fiber is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands. The ganglia include not just the sympathetic trunks but also the cervical ganglia (superior, middle, and inferior) that send sympathetic nerve fibers to the head and thorax organs and the celiac and mesenteric ganglia that send sympathetic fibers to the gut.

2. Innervation of the Kidneys

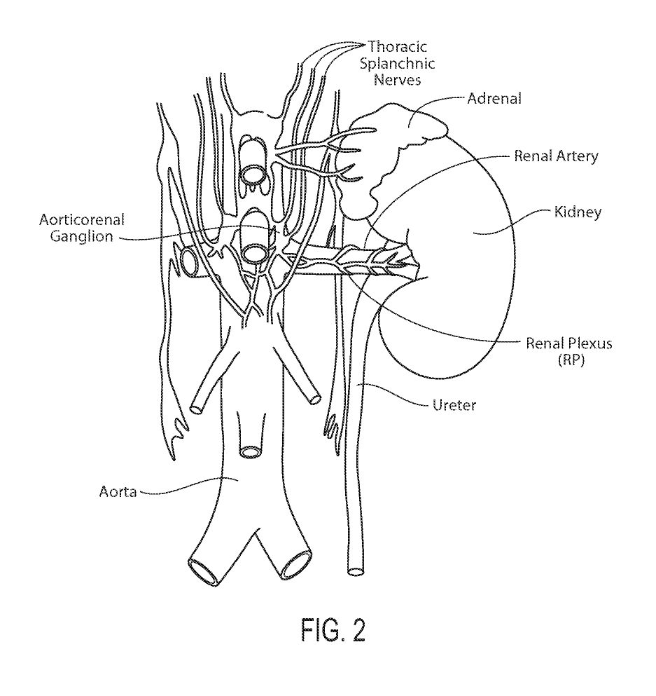

As FIG. 2 shows, the kidney is innervated by the renal plexus, which is intimately associated with the renal artery. The renal plexus is an autonomic plexus that surrounds the renal artery and is embedded within the adventitia of the renal artery. The renal plexus extends along the renal artery until it arrives at the substance of the kidney. Fibers contributing to the renal plexus arise from the celiac ganglion, the superior mesenteric ganglion, the aorticorenal ganglion, and the aortic plexus. The renal plexus, also referred to as the renal nerve, is predominantly composed of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, the first lumbar splanchnic nerve, and the second lumbar splanchnic nerve and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus and are distributed to the renal vasculature.

3. Renal Sympathetic Neural Activity

Messages travel through the SNS in a bidirectional flow. Efferent messages may trigger changes in different parts of the body simultaneously. For example, the SNS may accelerate heart rate, widen bronchial passages, decrease motility (movement) of the large intestine, constrict blood vessels, increase peristalsis in the esophagus, cause pupil dilation, cause piloerection (goose bumps), cause perspiration (sweating), and raise blood pressure. Afferent messages carry signals from various organs and sensory receptors in the body to other organs and, particularly, to the brain.

Hypertension, heart failure, and chronic kidney disease are a few of many disease states that can result from or be exacerbated by chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a long-standing, but somewhat ineffective, approach for counteracting overactivity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma have revealed increased renal norepinephrine spillover rates in patients with essential hypertension, particularly in young hypertensive subjects. In concert with increased norepinephrine spillover from the heart, increased renal norepinephrine spillover is consistent with the hemodynamic profile typically seen in early hypertension and characterized by increased heart rate, cardiac output, and renovascular resistance. It is now known that essential hypertension is commonly neurogenic, often accompanied by pronounced SNS overactivity.

Cardio-renal sympathetic nerve overactivity can be particularly pronounced in heart failure, as demonstrated by an exaggerated increase of norepinephrine overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end-stage renal disease are generally characterized by heightened sympathetic nervous activation. In patients with end-stage renal disease, plasma levels of norepinephrine above the median have been demonstrated to be predictive for both all-cause death and death from cardiovascular disease. This is also true for patients suffering from diabetic or contrast-induced nephropathy. There is compelling evidence that suggests that sensory afferent signals originating from the diseased kidneys are major contributors to the initiation and sustainment of elevated central sympathetic outflow in this patient group, which can facilitate the occurrence of the well-known adverse consequences of chronic sympathetic overactivity, such as hypertension, left ventricular hypertrophy, ventricular arrhythmias, sudden cardiac death, insulin resistance, diabetes, and metabolic syndrome.

(i) Renal Sympathetic Efferent Nerve Activity

Sympathetic nerves to the kidneys terminate in the blood vessels, the juxtaglomerular apparatus, and the renal tubules. Stimulation of the renal sympathetic nerves causes increased renin release, increased sodium (Nat) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies have significant limitations including limited efficacy, compliance issues, side effects, and others.

(ii) Renal Sensory Afferent Nerve Activity

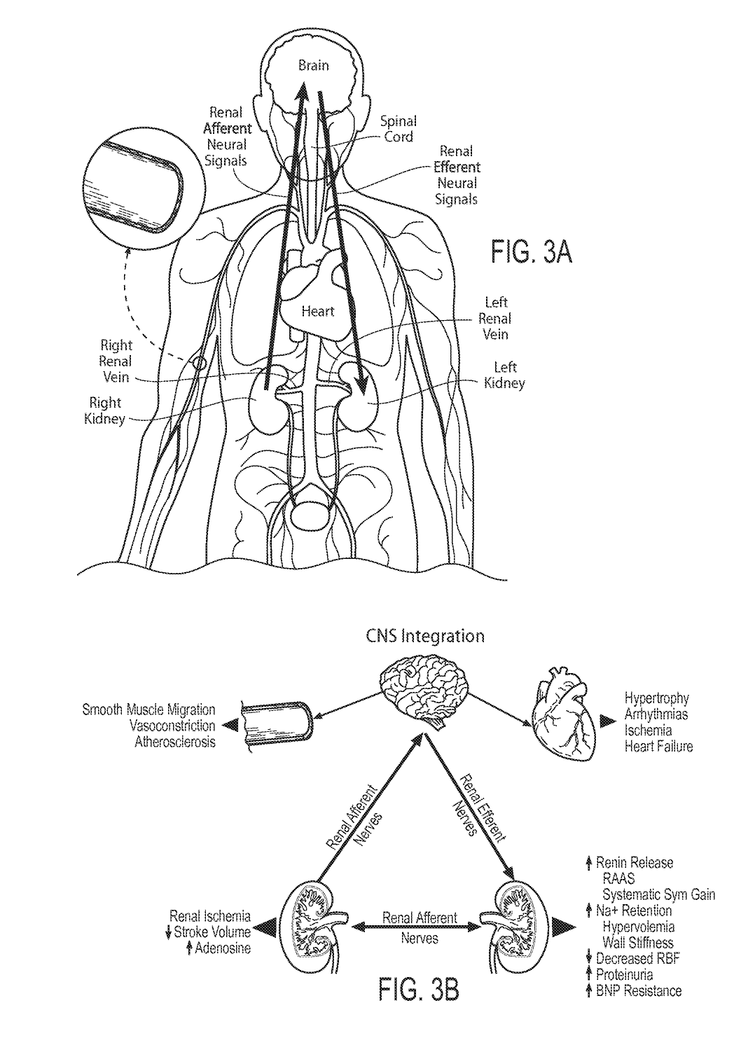

The kidneys communicate with integral structures in the CNS via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 3A and 3B, this afferent communication might be from the kidney to the brain or from one kidney to the other kidney (via the CNS). These afferent signals are centrally integrated and may result in increased sympathetic outflow. This sympathetic drive is directed toward the kidneys, thereby activating the RAAS and inducing increased renin secretion, sodium retention, volume retention, and vasoconstriction. Central sympathetic overactivity also impacts other organs and bodily structures innervated by sympathetic nerves, such as the heart and the peripheral vasculature, resulting in the described adverse effects of sympathetic activation, several aspects of which also contribute to a rise in blood pressure.

The physiology therefore suggests that (a) neuromodulation of tissue with efferent sympathetic nerves will reduce inappropriate renin release, sodium retention, and reduction of renal blood flow, and that (b) neuromodulation of tissue with afferent sensory nerves will reduce the systemic contribution to hypertension, and other disease states associated with increased central sympathetic tone through its direct effect on the posterior hypothalamus as well as the contralateral kidney. In addition to the central hypotensive effects of afferent renal neuromodulation, a desirable reduction of central sympathetic outflow to various other sympathetically innervated organs, such as the heart and the peripheral vasculature, is anticipated.

B. Additional Clinical Benefits of Renal Neuromodulation

As provided above, renal neuromodulation is likely to be valuable in the treatment of several clinical conditions characterized by increased overall, and particularly renal, sympathetic activity such as hypertension, metabolic syndrome, insulin resistance, diabetes, left ventricular hypertrophy, chronic and end-stage renal disease, inappropriate fluid retention in heart failure, cardio-renal syndrome, and sudden death. Since the reduction of afferent neural signals contributes to the systemic reduction of sympathetic tone/drive, renal neuromodulation may also be useful in treating other conditions associated with systemic sympathetic hyperactivity. Accordingly, renal neuromodulation may also benefit other organs and bodily structures innervated by sympathetic nerves, including those identified in FIG. 1. For example, a reduction in central sympathetic drive may reduce the insulin resistance that afflicts people with metabolic syndrome and Type II diabetes. Additionally, patients with osteoporosis are also sympathetically activated and might also benefit from the downregulation of sympathetic drive that accompanies renal neuromodulation.

C. Applying Energy to the Renal Artery

In accordance with the present disclosure, neuromodulation of a left and/or right renal plexus, which is intimately associated with a respective renal artery, may be achieved through application of energy (e.g., HIFU energy) from an extracorporeal energy source. The energy can be focused onto a desired location on or near the renal artery. In certain embodiments, the energy source may be used in conjunction with an energy-focusing mechanism that can be intracorporeal and, in certain embodiments, can be intravascular.

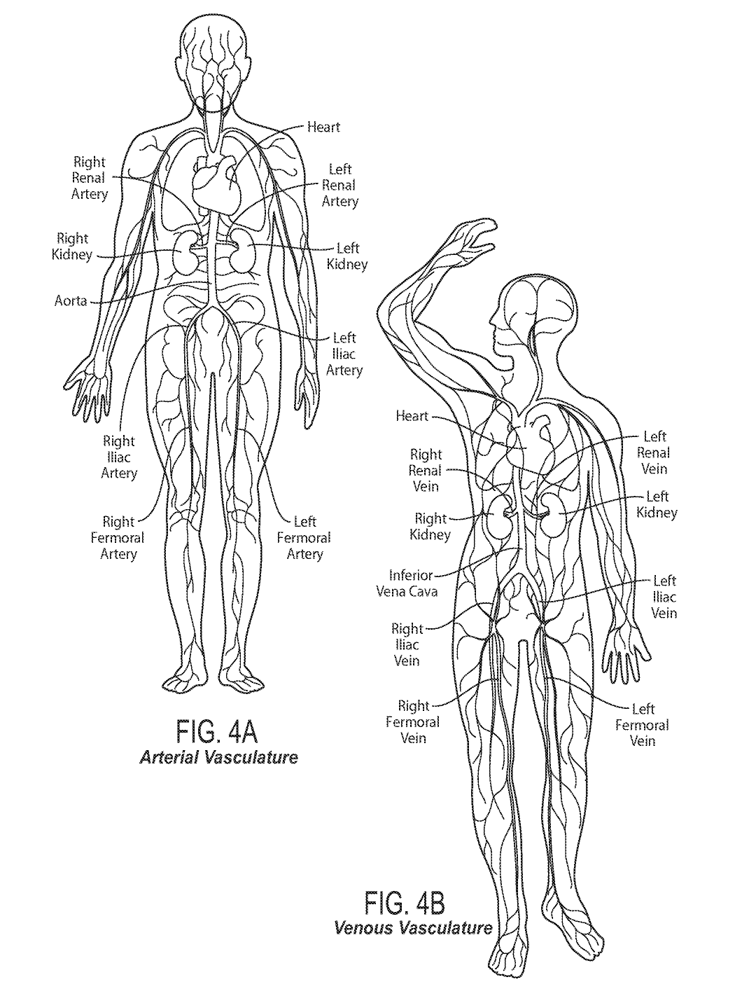

As FIG. 4A shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 4B shows, blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, blood is pumped through the right ventricle into the lungs, where it is oxygenated. From the lungs, oxygenated blood is conveyed into the left atrium. From the left atrium, oxygenated blood is conveyed by the left ventricle back to the aorta.

As described in greater detail below, in embodiments in which an HIFU energy source is used in conjunction with an intravascular focusing system, the focusing system may be introduced via the femoral artery, which may be accessed and cannulated at the base of the femoral triangle, just inferior to the midpoint of the inguinal ligament. A catheter or other carrying device for the focusing system may be inserted through this access site (e.g., percutaneously into the femoral artery) and passed into the iliac artery, the aorta, and either the left or right renal artery. This comprises an intravascular path that offers minimally invasive access to a respective renal artery and/or other renal blood vessels. Other suitable catheterization paths are also possible. For example, the wrist, upper arm, and shoulder region provide other locations for introduction of catheters into the arterial system. Catheterization of either the radial, brachial, or axillary artery may be utilized in select cases. Catheters introduced via these access points may be passed, for example, through the subclavian artery on the left side (or via the subclavian and brachiocephalic arteries on the right side), through the aortic arch, down the descending aorta, and into the renal arteries using suitable angiographic techniques.

D. Properties and Characteristics of the Renal Vasculature

Since neuromodulation of a left and/or right renal plexus may be achieved in accordance with the present disclosure through application of energy to the renal artery, properties and characteristics of the renal vasculature may impose constraints upon and/or inform the design of apparatus, systems, and methods for achieving such renal neuromodulation. Some of these properties and characteristics may vary across the patient population and/or within a specific patient over time, as well as in response to disease states, such as hypertension, chronic kidney disease, vascular disease, end-stage renal disease, insulin resistance, diabetes, metabolic syndrome, etc. These properties and characteristics, as explained below, may have bearing on the clinical aspects of treatment procedures and the specific designs of intravascular devices. Properties of interest may include, for example, material/mechanical, spatial, fluid dynamic/hemodynamic, and/or thermodynamic properties.

In certain embodiments, a device configured to be used with an external HIFU transmitter may be advanced percutaneously into or near either the left or right renal artery via a minimally invasive intravascular path. However, minimally invasive renal arterial access may be challenging because, as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, further complicating minimally invasive access. In particular, in embodiments in which the renal anatomy is characterized via imaging or other modalities, such factors may be considered in determining the appropriate focal point of HIFU energy. Significant variation among patients may be seen, for example, in relative tortuosity, diameter, length, and/or atherosclerotic plaque burden, as well as in the takeoff angle at which a renal artery branches from the aorta. The apparatus, systems, and methods for achieving renal neuromodulation via extracorporeal application of HIFU energy can account for these and other aspects of renal arterial anatomy and its variation across the patient population when applying energy to a renal artery.

In addition to complicating renal arterial access, specifics of the renal anatomy also can complicate a determination of one or more focal points for HIFU energy. When the neuromodulatory apparatus includes an ultrasound transducer, appropriate focusing of the energy on the vessel wall may be related to treatment success. In other embodiments, the positioning of the transducer(s) on the patient's skin relative to a renal artery or abdominal aorta may be considered. However, the application of energy through the patient may be impeded by intervening anatomical structures such as bone, cavities, or tissues. Ultrasound energy may interact differently with different types of structures. In particular, the presence of bones in the energy path may impede the transmission of ultrasound energy. As such, the positioning of the transducer may be selected to optimize the ultrasound path through the body. Furthermore, patient movement, respiration, and/or the cardiac cycle may cause significant movement of the renal artery relative to the aorta, and the cardiac cycle may transiently distend the renal artery (i.e., cause the wall of the artery to pulse), further complicating establishment of stable focal points for the HIFU energy.

Once the focal points on the artery have been established, the artery can be modulated via the neuromodulatory apparatus. As discussed in greater detail below, the intima-media thickness separating the vessel lumen from its adventitia means that target renal nerves may be multiple millimeters distant from the luminal surface of the artery. Sufficient energy can be delivered to the target renal nerves to modulate the target renal nerves without excessively heating and desiccating the vessel wall. Another potential clinical complication associated with excessive heating is thrombus formation from coagulating blood flowing through the artery. Given that this thrombus may cause a kidney infarct, thereby causing damage to the kidney, thermal treatment from within the renal artery can be applied carefully. Accordingly, the complex fluid mechanic and thermodynamic conditions present in the renal artery during treatment, particularly those that may impact heat transfer dynamics at the treatment site, may be important in applying energy (e.g., in applying thermal energy from within the renal artery).

The HIFU-application device may also be configured to allow for adjustable positioning and repositioning of the ultrasound energy focal points proximate to or within the renal artery since location of treatment may also impact clinical aspects of a procedure. Such repositioning may involve one or both of adjustment of the extracorporeal HIFU-application device or an internal-focusing or energy-targeting system. In certain embodiments, it may be tempting to apply a full-circumferential treatment given that the renal nerves may be spaced circumferentially around a renal artery. However, in some instances, the full-circle lesion likely resulting from a continuous circumferential treatment may create a heightened risk of renal-artery stenosis, thereby negating any potential therapeutic benefit of the renal neuromodulation. Therefore, the formation of more complex lesions along a longitudinal dimension of the renal artery (e.g., in a linear pattern or a continuous or non-continuous helical pattern) and/or repositioning of the neuromodulatory apparatus to multiple treatment locations may be desirable. It should be noted, however, that a benefit of creating a circumferential ablation may outweigh the risk of renal-artery stenosis or the risk may be mitigated with certain embodiments or in certain patients and creating a circumferential ablation can be a goal. Additionally, variable positioning and repositioning of the focal points of the neuromodulatory apparatus may prove to be useful in circumstances where the renal artery is particularly tortuous or where there are proximal branch vessels off the renal-artery main vessel, making treatment in certain locations challenging. Manipulation of any energy-focusing device in a renal artery can also consider mechanical injury imposed by the device on the renal artery. Motion of a device in an artery, for example by inserting, manipulating, negotiating bends, and so forth, may cause mechanical injury such as dissection, perforation, denuding of the intima, or disruption of the interior elastic lamina.

Further, depending on the structure of the focusing device, blood flow through a renal artery may be temporarily occluded for a short time with minimal or no complications. Occluding blood flow may or may not be desirable. For example, in some cases, partially or fully occluding blood flow can enhance the heating of tissue at a target site by reducing or eliminating heat loss to the blood. This may enhance the strength and/or predictability of the heating. In other cases, as described in greater detail below, allowing blood flow through a target site during treatment can be useful to reduce heating of non-target tissue (e.g., at an inner luminal wall of a blood vessel). However, occlusion for a significant amount of time may cause injury to the kidney such as ischemia. In some cases, it can be beneficial to avoid occlusion if occlusion is a consequence of an embodiment. Furthermore, the duration of occlusion can be limited (e.g., to no more than about three or four minutes).

Based on the above-described challenges of (1) extracorporeal HIFU-energy application, (2) focusing the energy at the appropriate focal point or focal zone on or near the renal artery, (3) positioning and potentially repositioning the focal points for multiple treatment locations, (4) imaging the renal artery, and (5) avoiding or limiting duration of blood flow occlusion, various independent and dependent properties of the renal vasculature that may be of interest include, but are not limited to, vessel diameter, length, intima-media thickness, coefficient of friction and tortuosity; distensibility, stiffness and modulus of elasticity of the vessel wall; peak-systolic and end-diastolic blood flow velocity, as well as the mean systolic-diastolic peak blood flow velocity, and mean/max volumetric blood flow rate; specific heat capacity of blood and/or of the vessel wall, thermal conductivity of blood and/or of the vessel wall, thermal convectivity of blood flow past a vessel wall treatment site and/or radiative heat transfer; and renal artery motion relative to the aorta, induced by respiration, patient movement, and/or blood flow pulsatility, as well as the takeoff angle of a renal artery relative to the aorta. These properties will be discussed in greater detail with respect to the renal arteries. However, depending on the apparatus, systems, and methods utilized to achieve renal neuromodulation, such properties of the renal arteries also may guide and/or constrain design characteristics, energy-focusing systems, or various imaging modalities.

In embodiments in which an accessory focusing system or device (e.g., a catheter) is positioned within a renal artery, the device can conform to the geometry of the artery. Renal artery vessel diameter, D_{RA}, typically is in a range of about 2-10 mm, with an average of about 6 mm. Renal artery vessel length, L_{RA}, between the ostium at the aorta/renal artery juncture and its distal branchings, generally is in a range of about 5-70 mm (e.g., in a range of about 20-50 mm). Since the target renal plexus is embedded within the adventitia of the renal artery, the composite intima-media thickness, IMT (i.e., the radial outward distance from the artery's luminal surface to the adventitia containing target neural structures), also is notable and generally is in a range of about 0.5-2.5 mm, with an average of about 1.5 mm. Although a certain depth of treatment is important to reach the target neural fibers, the depth can be limited (e.g., to less than about 5 mm from an inner wall of the renal artery) to avoid non-target tissue and anatomical structures such as the renal vein.

An additional property of the renal artery that may be of interest is the degree of renal motion relative to the aorta, induced by respiration and/or blood flow pulsatility. A patient's kidney, located at the distal end of the renal artery, may move as much as four inches cranially with respiratory excursion. This may impart significant motion to the renal artery connecting the aorta and the kidney. Accordingly, a balance of stiffness and flexibility can be useful in the neuromodulatory apparatus to maintain contact between a thermal-treatment element and the vessel wall during cycles of respiration. Furthermore, the takeoff angle between the renal artery and the aorta may vary significantly between patients, and also may vary dynamically within a patient (e.g., due to kidney motion). The takeoff angle generally may be in a range of about 30°-135°.

These and other properties of the renal vasculature may impose constraints upon and/or inform the design of apparatus, systems, and methods for achieving renal neuromodulation via intravascular access. Some embodiments can be configured for accessing the renal artery, facilitating stable contact between neuromodulatory apparatus and a luminal surface or wall of the renal artery, and/or modulating the renal nerves with the neuromodulatory apparatus.

### III. APPARATUSES, SYSTEMS, AND METHODS FOR RENAL NEUROMODULATION

A. Overview

The representative embodiments provided herein include features that may be combined with one another and with the features of other disclosed embodiments. In an effort to provide a concise description of these embodiments, not all features of an actual implementation are described in the specification. It should be appreciated that in the development of any such actual implementation, as in any engineering or design project, numerous implementation-specific decisions can be made to achieve the developers' specific goals, such as compliance with system-related and business-related constraints, which may vary from one implementation to another. Further, as appropriate, the features of various embodiments may be exchanged or used in combination with one another.

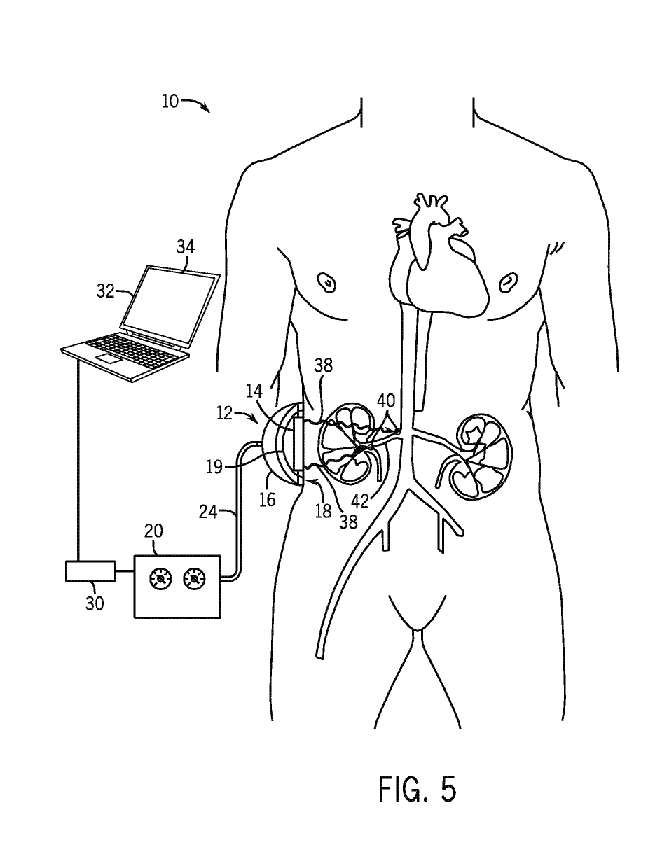

FIG. 5 shows a system 10 for inducing neuromodulation of a left and/or right renal plexus through application of HIFU energy. As described herein, the left and/or right renal plexus surrounds the respective left and/or right renal artery. The renal plexus extends in intimate association with the respective renal artery into the substance of the kidney. The system 10 induces neuromodulation of a renal plexus by extracorporeal application of HIFU energy.

The system 10 includes a HIFU treatment device 12 that can be configured to transmit acoustic energy via a transducer 14. As depicted, the transducer 14 may be located within a housing 16 that is suitably sized and shaped to be applied to a patient's skin. For example, the contact surface 18 of the housing 16 may be curved or shaped to facilitate direct and stable contact with the patient. Furthermore, the housing 16 may include appropriate structures for transmission of HIFU energy. In embodiments in which the transducer 14 is sealed or covered by an aperture cover, the cover can be formed from a material that is capable of transmitting the ultrasound energy. The transducer 14 may refer to a single transducer or a plurality of transducers 14.

The HIFU treatment device 12 can be coupled to an acoustic energy source (e.g., a HIFU energy generator 20). Under the control of the caregiver and/or an automated control algorithm 30, the generator 20 can generate a selected form and magnitude of acoustic energy (e.g., a particular energy frequency and/or magnitude). A cable 24 can connect the transducer 14 to the generator 20. In some embodiments, energy delivery via the HIFU treatment device 12 can be timed to at least generally correspond to one or more body cycles (e.g., respiration and/or heart beat). For example, a body cycle can be monitored, corresponding changes in the position of an artery can be predicted, and energy can be delivered at certain intervals within the cycle to increase energy delivery to targeted tissue and/or to reduce energy delivery to non-targeted tissue.

The generator 20 may be part of a processing device 32 that may include processing circuitry, such as a microprocessor and a display 34. The processing circuitry may be configured to execute stored instructions relating to the control algorithm 30. The processing device 32 may be configured to communicate with the HIFU treatment device 12, for example, via the cable 24, to control power to the transducer 14 and/or to obtain signals from the transducer 14 or any associated sensors. The processing device 32 (e.g., via the display 34) may be configured to provide indications of power levels or sensor data, such as audio, visual, or other indications, or may be configured to communicate the information to another device.

In certain embodiments, the HIFU treatment device 12 may be configured to be placed directly on the exterior surface of the patient, e.g., on the skin. In this manner, the HIFU treatment device 12 can be noninvasive. Depending on the patient's clinical condition, the system 10 may include appropriate positioning devices for orienting the patient relative to the system 10. Such devices may include beds and mounting systems for the system 10. In particular, because the transducer 14 can be configured to transmit HIFU energy through the patient's tissue and bone, it may be advantageous to position the HIFU treatment device 12 at a desired location on the skin relative to intervening anatomical structures (e.g., the pubic bone or spine) that may interfere with appropriate transmission of HIFU energy. Further, the desired location of the HIFU treatment device 12 may be determined in conjunction with imaging or other treatment-location targeting modalities. For example, the HIFU treatment device 12 may be moved along the patient's tissue until the desired location is achieved (e.g., until an appropriate signal strength or imaging brightness is achieved). Hybrid techniques that merge ultrasound images with MRI images to find the best window may also be used. For example, previously acquired images from an MRI system can be correlated with the ultrasound images to determine aspects of treatment.

The HIFU treatment device 12 may be used in conjunction with techniques and systems for location of the renal-artery neuromodulation treatment area and targeting of the HIFU energy on the treatment area. Such techniques may be extracorporeal or intracorporeal, as discussed herein. Once the HIFU energy is appropriately focused, the purposeful application of energy from the generator 20 to tissue by the transducer 14 can induce one or more desired neuromodulating effects on localized regions of a renal artery 42 and adjacent regions of the renal plexus, which lay intimately within, adjacent to, or in close proximity to the adventitia of the renal artery 42. For example, the purposeful application of the energy may achieve neuromodulation along all or a portion of the renal plexus.

Treatment may include application of focused ultrasound energy to achieve sustained heating, sonication, and/or cavitation. Desired thermal heating effects may include raising the temperature of target neural fibers above a desired threshold to achieve non-ablative thermal alteration, or above a higher temperature to achieve ablative thermal alteration. For example, the target temperature may be above body temperature (e.g., approximately 37° C.) but less than about 45° C. for non-ablative thermal alteration, or the target temperature may be about 45° C. or higher for ablative thermal alteration.

The HIFU treatment device 12 can be equipped with the transducer 14, which may be any appropriate device for transmitting ultrasound energy having suitable characteristics (e.g., frequencies). In some embodiments, for example, the transducer 14 may be an ultrasonic crystal. The HIFU treatment device 12 may include an integral focusing structure, such as a convex acoustic mirror in the form of a convex hemispheric cavity configured to focus ultrasound waves (shown as arrows 38) on focal points 40. The geometry of the HIFU treatment device 12 may be such that when the transducer 14 is placed in contact with the skin at a suitable location, the ultrasound waves form one or more focal points at or near the renal artery 42. Such focusing may be accomplished, for example, with the help of additional devices or focusing systems. In some embodiments, the focal points 40 can be located in an adventitial layer or slightly beyond.

It is expected that, in certain embodiments, the ultrasound intensity for ablation of renal nerves may, for example, be in the range of 1 to 4 kW/cm² and may be delivered for a total of 10 to 60 sec to create one focal lesion. The treatment parameters of time and energy may be interrelated based on the properties of the tissue being treated as well as the intervening tissues. For example, a longer treatment time at lower energy (or, conversely, a shorter treatment time at higher energy) may achieve similar results. The exact parameters for sonication may be established in a series of animal experiments for the selected design of the HIFU crystal and mirror. The selected parameters may be chosen, for example, to disable conduction of renal nerves for at least several months while causing reduced or minimal damage to surrounding tissue.

The transducer 14 can be coupled by electrical connectors to the generator (e.g., generator 20 shown in FIG. 5) that can deliver electric excitation to the crystal making it vibrate with the desired frequency and intensity. It should be appreciated that, as discussed herein, the transducers 14 may be depicted as a single transducer, but the transducers 14 may be multiple transducers or an array of transducers. Further, the transducers 14 may be formed in a variety of shapes, such as cylindrical, rectangular, or elliptical, and may have some degree of freedom of motion to allow for articulation relative to the housing 16. For example, tilt of the transducers 14 may be controlled by a steering mechanism (e.g., a manual or micromechanical steering mechanism). The transducer 14 tilt can be used to change the shape of a lesion. In addition, the transducer 14 may be oriented in any suitable orientation relative to the housing 16. It some embodiments, it is appreciated that several transducers 14 (e.g., sonicating crystals or several cavities in one crystal) may be mounted on a single HIFU treatment device 12 to speed up energy delivery (e.g., sonication). In this case, focusing (e.g., parabolic) mirrors may be arranged in a desired angle to create overlapping lesions. In particular embodiments, energy can emanate from multiple surfaces of the transducer 14 to create multiple potential focal points 40.

Further, the transducer 14 may by a single-element or multi-element transducer that can be side-looking or forward-looking. In addition to these designs, the transducer 14 may also image and deliver therapy (e.g., as discussed herein with respect to certain embodiments). In other embodiments, the HIFU treatment device 12 may include a second transducer dedicated to imaging functions. The imaging transducer may be designed in such a way that it is highly reflective to the therapy frequency yet transparent to the imaging frequency. Furthermore, the shape of the imaging transducer may be used to focus the reflected therapy energy. The choice of acoustic materials, impedance, and thickness, as well as the design of the electrical circuit connected to the imaging transducer may also affect focusing. The therapy portion of the transducer 14 may be a single-element or multi-element transducer that can be a partial cylinder or full cylinder with a mechanical focus in the height and/or circumferential direction.

B. Extracorporeal HIFU Devices with Internal Targeting Systems

The system 10 (e.g., as shown in FIG. 5) may be used in conjunction with internal targeting systems for locating or specifying an intended target region.

1. Intravascular-Based Technologies

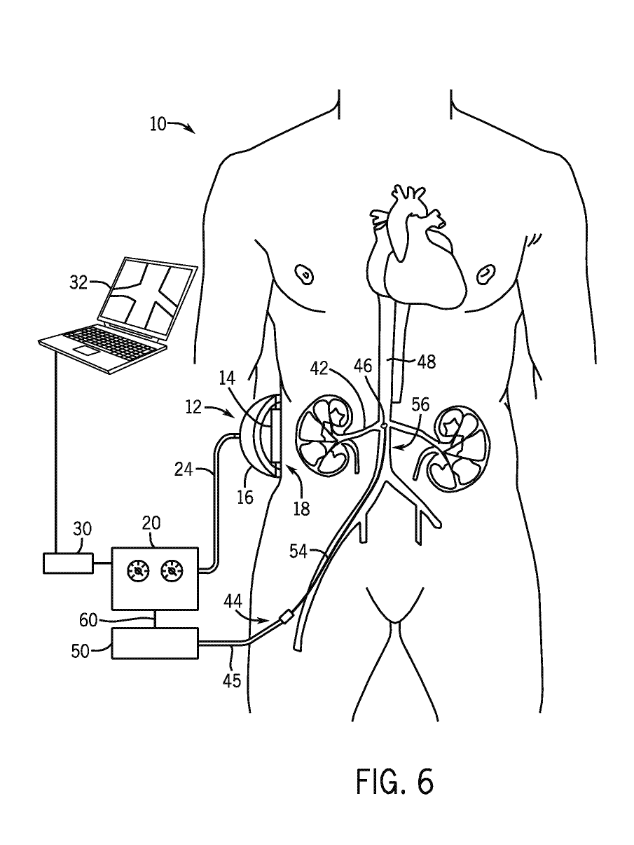

In some embodiments, for example, an intravascular imaging agent may be used in conjunction with the appropriate imaging technology to provide an image of the renal artery to facilitate focusing of the HIFU energy. In other embodiments, the imaging technology may employ an intravascular device 44 (e.g., as shown in FIG. 6). In certain embodiments, the intravascular device 44 can carry a targeting element 46 for providing information about the renal artery that may be used for focusing the transducer 14 on the appropriate treatment sites. Furthermore, a pre-treatment image can be obtained (e.g., using intravascular ultrasound, optical coherence tomography, or another suitable imaging modality). The pre-treatment image can then be registered to a suitable reference (e.g., an anatomical or non-anatomical reference). A suitable reference can be a stationary or non-stationary feature having a known location. In some cases, the known location can be based on a known static or dynamic correlation relative to another feature. As described in greater detail below, pre-treatment images can be used to facilitate positioning the intravascular device 44.

After the treatment site is located, the external HIFU treatment device 12 can be placed on the patient. The next stage can be to locate the intended treatment site relative to the HIFU treatment device 12 as well as to verify that the acoustic path to the target from the HIFU treatment device 12 is acceptable. Coordinate transformations may be used to locate the target relative to the HIFU treatment device 12. However, there are a variety of different energy modalities that can be used to communicate the internal position to the external HIFU treatment device 12. In certain embodiments, the modalities included in the targeting element 46 may encompass devices that facilitate ultrasound, magnetic targeting, acoustic targeting, and Doppler techniques as discussed herein. However, it should be understood that the disclosure encompasses additional types of targeting technologies for providing information about the renal artery and/or for focusing HIFU energy on the appropriate focal points. Further, it should be understood that, in the exemplary embodiments, the locations of transmitters and receivers may be exchanged. An acoustic contrast agent may be used during treatment to help maintain the focus of the HIFU treatment device 12 on the renal artery.

The intravascular device 44 as provided herein may be used in conjunction with any appropriate internal targeting systems. Furthermore, the intravascular device 44 may be configured to access the renal artery from an intravascular pathway originating in the femoral artery. To that end, the intravascular device 44 may be coupled by a cable 45 to an elongated shaft 54 that can be specially sized and configured to accommodate passage through an intravascular path, which leads from a percutaneous access site in, for example, the femoral, brachial, radial, or axillary artery to a targeted treatment site within a renal artery. In this way, the caregiver can be able to orient the intravascular device 44 within the aorta 48 or the renal artery 42 for its intended purpose. For example, the intravascular device 44 can be designed to insert the targeting element 46 at an appropriate location relative to the renal artery 42, the desired focal points 40, and the HIFU treatment device 12 so that the ultrasound energy may be focused on the focal points 40. The targeting element 46 may be any appropriate element that can be configured to be carried by the inserted intravascular device 44 and may include sensors or energy transmitters and/or receivers as desired. The targeting element 46 can be capable of providing information related to the geometry or location of the renal artery 42 or the focal points 40.

The intravascular device 44 may be inserted into the patient by manipulating a handle assembly 50 from outside the intravascular path, and the caregiver may advance the elongated shaft 54 through the tortuous intravascular path, including the aorta 48 and the renal artery 42, and remotely manipulate or actuate a distal end region 56. The handle assembly 50 may include an actuatable element, such as a knob, pin, or lever that may control flexing of the elongated shaft 54 within the vasculature. Further, the elongated shaft 54 may flex in a substantial fashion to gain entrance into a respective left/right renal artery by manipulation of the elongated shaft 54. In some embodiments, the flexing may be imparted by a guide catheter, such as a renal guide catheter with a preformed or steerable bend near the distal end region 56 that can direct the elongated shaft 54 along a desired path such as from an aorta to a renal artery. In other embodiments, the flexing may be imparted by a guidewire that can be first delivered into a renal artery. The elongated body 54 can comprise a guidewire lumen that can be then passed over the guidewire into the renal artery. Alternatively, following insertion of a guidewire into a renal artery, a delivery sheath may be passed over the guidewire (e.g., a lumen defined by the delivery sheath can slide over the guidewire) into the renal artery, for example using a 6 French opening size. Once the delivery sheath is placed in the renal artery, the guidewire may be removed and a treatment catheter may be delivered into the renal artery. Furthermore, in particular embodiments, the flexing may be controlled via the handle assembly 50, for example, by an actuatable element or by another control element. In particular, the flexing of the elongated shaft 54 may be accomplished as provided in U.S. patent application Ser. No. 12/545,648, “Apparatus, Systems, and Methods for Achieving Intravascular, Thermally-Induced Renal Neuromodulation” to Wu et al., which is incorporated by reference in its entirety herein for all purposes. Any one of the embodiments of the system 10 that includes the intravascular device 44 described herein may be delivered over a guidewire using conventional over-the-wire techniques. When delivered in this manner, the elongated shaft 54 can include a passage or lumen accommodating passage of a guidewire.

The intravascular devices 44 may carry targeting elements 46 designed to be side-looking (e.g., directed toward the artery wall) or forward-looking. In either case, the goal of using the intravascular devices 44 can be to locate the particular treatment region in the renal artery 42. After the treatment site is located, the external HIFU treatment device 12 can be placed on or near the patient. The next stage can be to locate the intended treatment site relative to the HIFU treatment device 12 as well as verify that the acoustic path to the target from the HIFU treatment device 12 is acceptable. Coordinate transformations can be used to locate the target relative to the HIFU treatment device 12. However, there are a variety of different modalities (e.g., energy sources) that can be used to communicate the internal position to the external HIFU device as described herein.

2. Internal Magnetic Targeting and Tracking

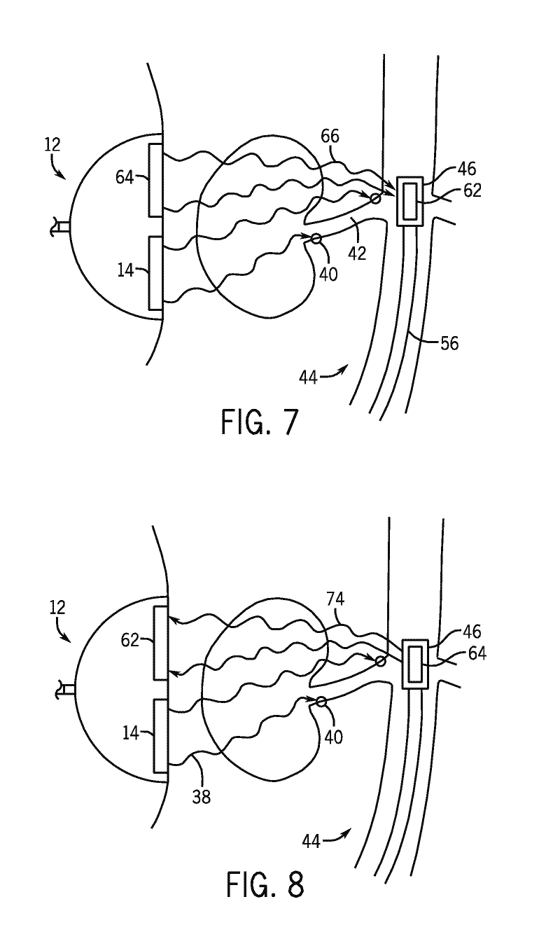

In some embodiments (e.g., as shown in FIG. 7), magnetic fields can be used to communicate the position of the intended target (e.g., an appropriate focal point 40) relative to the HIFU treatment device 12. For example, a magnetic receiver 62 may be located on a targeting element 46 associated with the distal end region 56 of the intravascular device 44. A magnetic transmitter 64 may be located on the HIFU treatment device 12 and configured to transmit magnetic energy (e.g., as shown by arrows 66) to the magnetic receiver 62. It should be understood that the magnetic receiver 62 need not be located directly at the target location when the distance and direction of the target location from the sensor are known. In particular embodiments, a plurality of magnetic sensors may be beneficial for target localization. A magnetic receiver 62 may also be located on the HIFU treatment device 12 if a common transmitter is used. If the distance of the magnetic receiver 62 from the treatment location for imaging is known, then the focal point 40 of the HIFU treatment device 12 may be determined. In embodiments in which the size of the magnetic receiver 62 is larger than a diameter of the renal artery 42, it may not be possible to locate the magnetic receiver 62 directly within the therapy aperture. In such embodiments, the control algorithm 30 (FIG. 6) may employ a transformation to account for the estimated distance from the magnetic receiver 62 to the HIFU aperture assuming a rigid body. The transformation may be used for focusing the HIFU energy (e.g., as shown by arrows 38 in FIG. 5) on the desired focal points 40. For example, the acoustic coordinates of the magnetic receiver 62, which sees the treatment site, can be transformed to a magnetic coordinate system of the magnetic receiver 62 and to a magnetic coordinate system of the HIFU treatment device 12, and finally transformed to the acoustic coordinate system of the HIFU treatment device 12, which can be used for the treatment. Further, the intravascular device 44 and targeting element 46 may be designed (e.g., via geometry and shape) to fit at the opening or junction of the renal artery 42 and the aorta, which may reduce the variability for the transformation.

FIG. 8 shows an alternative arrangement in which the magnetic transmitter 64 can be located on the targeting element 46 and the magnetic receiver 62 can be coupled to the HIFU treatment device 12. Accordingly, communication (e.g., as shown by arrows 74) can be from the intravascular device 44 to the HIFU treatment device 12. Generally, the magnetic transmitter 64 is larger than the magnetic receiver 62. However, given the possible close proximity of the HIFU treatment device 12 to the intravascular device 44, a low-energy magnetic transmitter 64 may be used for the HIFU treatment device 12. In this case, the implementation can be similar to the preceding example. The translation vector and possible rotation between the magnetic transmitter 64 and magnetic receiver 62 can be known using the magnetic field. Since the magnetic receiver 62 now sits on the HIFU treatment device 12, a transformation from the magnetic receiver coordinate system to the acoustic coordinate system of the HIFU treatment device 12 may be performed. Appropriate transformations may include, but are not limited to, the Euler rotation matrix or quaternion notation, as well as translation vectors. Using the information from the magnetic receiver 62 and transmitter communication, the HIFU treatment device 12 may be focused on the appropriate focal point 40 and HIFU energy (e.g., as shown by arrows 38) may be used to modulate the renal nerves.

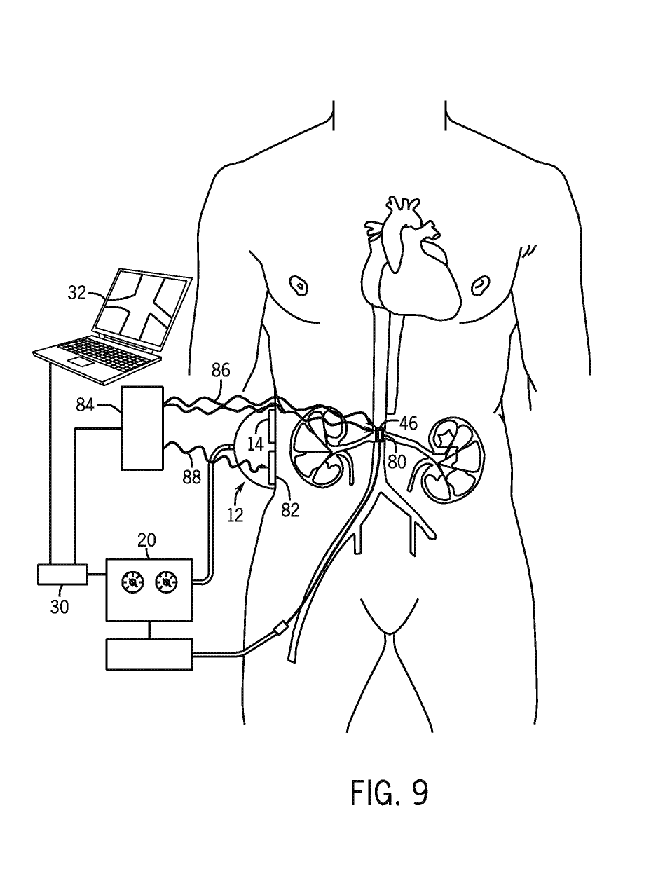

An alternative design (e.g., as shown in FIG. 9) may include two magnetic receivers (e.g., shown as a receiver 80 associated with the targeting element 46 and a receiver 82 associated with the HIFU treatment device 12 in FIG. 9) with a separate magnetic transmitter 84 placed outside but near the patient. Magnetic transmitters can be relatively heavy because of the fields that need to be generated and power consumption. The size of the transmitter 84 may make it ergonomically impractical to mount it on the HIFU treatment device 12. A suitable separate location may be, for example, on the patient bed or on a stand near the patient. In this case, the transmitter 84 can act as the anchor between the intravascular device coordinate system and the HIFU treatment device coordinate system and can communicate with receiver 80 (e.g. as shown by arrow 86) and receiver 82 (e.g. as shown by arrow 88). The transmitter 84 can allow the translation and rotation of each magnetic receiver to be determined. Therefore, the coordinate systems of the two magnetic receivers can be related to focus on the desired focal points.

3. Internal Acoustic Targeting and Tracking

After the intended target is located, the location can be communicated to the processing device 32 and used for focusing the HIFU treatment device 12. For example, logic can be used to determine if the location of the HIFU treatment device 12 relative to the target is acceptable for treatment. If not, then the system 10 may guide the physician to reposition the HIFU treatment device 12. Accordingly, the HIFU treatment device 12 may provide location information to the processing device 32.

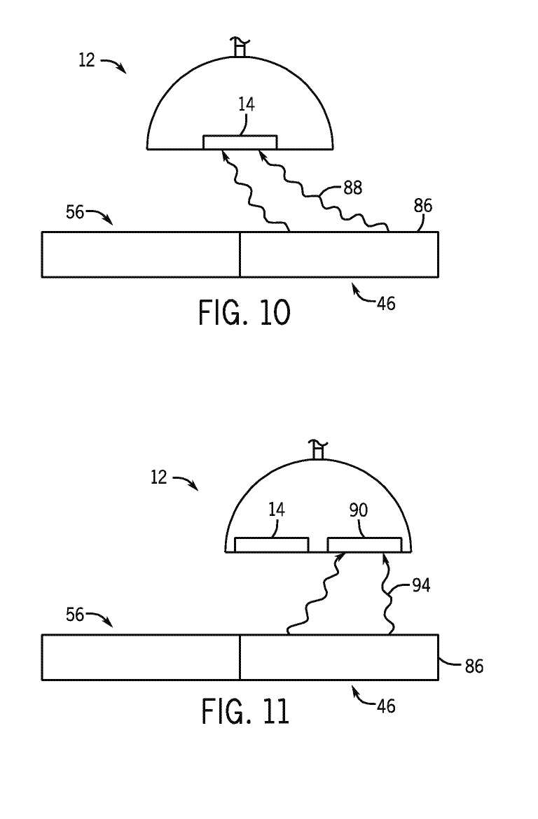

When the intravascular device 44 has an acoustic transmitter (e.g., the transmitter 86 shown in FIG. 10), it can be possible to communicate a position back to the HIFU treatment device 12 acoustically. For example, an acoustic pulse (e.g., as shown by arrows 88) can be sent from the transmitter 86. Element(s) within the HIFU treatment device 12 may be used to detect the pulse from the intravascular device 44. These transducer elements (e.g., transducer 14) in the HIFU treatment device 12 may also be used for therapy or may be specially configured as receive elements. For example, FIG. 11 depicts an embodiment in which the HIFU treatment device 12 can include a dedicated acoustical receiver 90 for receiving transmissions (e.g., as shown by arrows 94) to locate the intravascular device 44 within the body. The process of locating the target using an acoustic pulse can be dependent on the element type used in the therapy device for detection. For example, the process may be directional or omni-directional.

In some embodiments, individual elements within the HIFU treatment device 12 may be configured to detect an incoming pulse. When the acoustic energy is first observed by the receive element (e.g., transducer 14 or receiver 90), it can define an acoustic time that is governed by the velocity of tissue (e.g., 1540 m/sec) and the distance to the target. In some cases, the individual elements may be significantly larger than the wavelength of transmission in one or more dimensions. That is, a larger element can increase the directionality of the receiver while a smaller element can decrease the directionality. In this case, the acoustic pulse may be located using signal processing methods. In another method, the larger element may have an electrical/mechanical focus to even further define the direction. In some embodiments, locating the acoustic pulse when energy is first detected may not be sufficient to determine the distance to the target. Correlation methods or integration methods may be employed to locate the ‘center’ of the pulse. For elements that are directional, alignment may be a factor. The directionality may be addressed by moving the external HIFU treatment device 12 to find a stronger signal. Another method can include use of a multi-element structure (e.g., two transducers that are electronically delayed and that provide more information than a single transducer). In some embodiments, one or more electromagnetic sensors (e.g., coil sensors) can be used intravascularly in conjunction with an extracorporeal electromagnetic generator to provide location information. For example, a plurality of electromagnetic sensors can be included on an intravascular device in an orthogonal configuration. A processing device operably connected to the sensors can be configured to receive signals from the sensors and to generate location information for the intravascular device in three dimensions based on the signals.

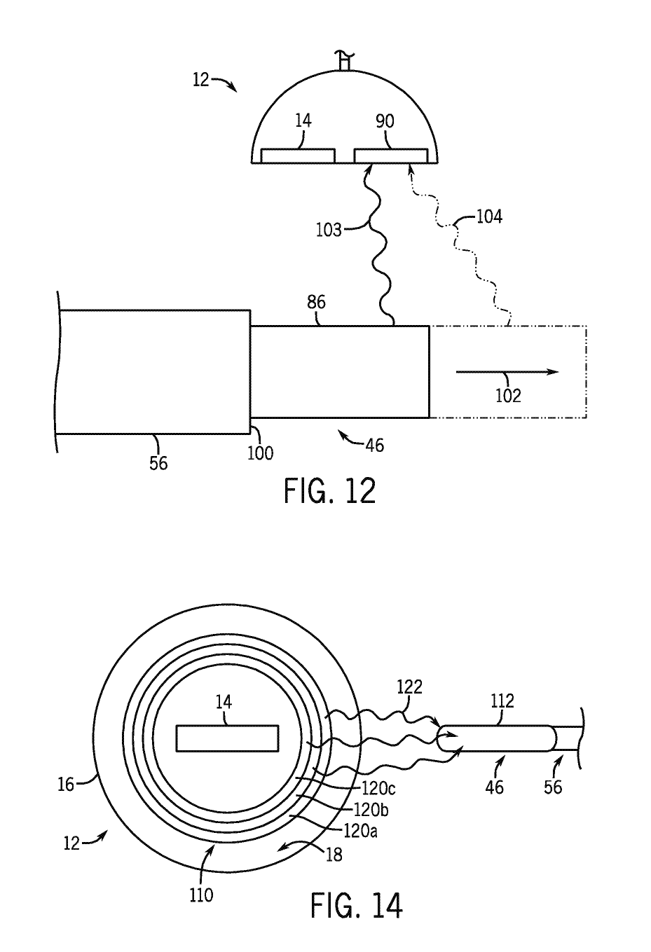

In some embodiments, the HIFU treatment device 12 can be a focused piston or a single-element device (e.g., as shown in FIG. 12). In the depicted embodiment, the transmitter 86 can be configured to move axially along arrow 102 by moving relative to the distal end region 56 of the vascular device. For example, the piston movement may be movement within a chamber 100 formed in the distal end region 56. As a result, the transmission path to the receiver 90 can change (e.g., as shown by arrows 103 and 104). Feedback may be used to place the HIFU treatment device 12 directly above the intended target even though it is a single large element. Movement of the HIFU treatment device 12 away from the transmitter 86, can cause the pulse length to increase and the peak intensity to decrease in a manner that depends on the directionality of the devices, frequency response of the devices, distances involved, attenuation of tissue, transmit pulse frequency, etc. This signature can be used to optimize the location of the transducer 14 above the intended target. Alternatively, rather than an actuating or piston-type transmitter 86, the entire intravascular device 44 may be manipulated within the vasculature to move the transmitter 86. Further, as noted, the arrangement of the transmitter 86 and receiver 90 may be exchanged.

In certain embodiments, an annular-array transducer 14 may be used for therapy. An annular array can electronically focus acoustic energy along the depth axis. In this case, the arrival time, the spread, the frequency, and the intensity of the energy may also be used to locate the transmitter along the intended HIFU beam axis (e.g., the acoustic pulse may help guide the physician to place the HIFU treatment device 12 directly on top of the transmitter 86). It should be noted that it is not necessary for the transmitter 86 to be directly located at the intended treatment region. If the distance and direction of the intended target from the transmitter 86 is known, then the targeting system may be used to compensate for the position of the transmitter 86.

If the elements in the HIFU treatment device 12 are smaller than a wavelength in all dimensions, then the arrival time of the transmitted pulse can be used to help guide the physician to move the therapy transducer such that the target may be treated. The arrival time information can also be helpful in developing the phase or delay profile for treatment if the device is at the location of treatment. For example, phases of the HIFU treatment device 12 may be set to minimize the error with the actual received pulse.

Use of an imaging array in the HIFU treatment device 12 may also be helpful. The array may be part of a transmitter or a receiver of the intravascular device. A point target on the external device can send out a sound wave or pulse to an array on the intravascular receive end, which can measure the intensity of the received sound wave. Further, the receive end of the intravascular device can ping back. That is, the external and internal devices can have two-way handshake communication. This may be beneficial for targeting. In some embodiments, the intravascular device can send a transmission that can be received by the external HIFU treatment device 12. The HIFU treatment device 12 may produce an image based on the received signal. Based on the intensity, a particular tissue area may be targeted. In this case, the pulsing of the transmitter can be synchronized with the “imaging” of a line or particular field of view. In this manner, the transmit element can be “imaged” and, depending on the brightness or overall spot size, can suggest where the intended target is relative to the HIFU treatment device 12. Further, if the HIFU treatment device 12 has a multi-element transducer, then knowing “when” the device transmitted (e.g., time-stamp information) can help with mapping the distance to the transmitter. The arrival times can be used to determine the location relative to the array. Depending on the array type, knowledge of the actual location of the transmitter may be determined by comparing two array locations. The intravascular device may locate a treatment area not immediately adjacent to the renal artery. Acoustic transmitters/receivers on the intravascular transducer can be used to communicate the intravascular-device location to the HIFU treatment device 12. In this case, the transformation can be from the acoustic coordinate system of the intravascular transducer to the acoustic coordinate system of the transmitter/receiver on the intravascular transducer to the external HIFU treatment device 12. This can be a three-stage transformation between different acoustic coordinate systems, and the result can be targeting information for the HIFU treatment device 12 to target the renal artery.

The transmitter may also be used to obtain estimates of tissue attenuation (e.g., the relative reduction in amplitude of an ultrasound beam as a function of distance through the tissue) and the required electrical power for treatment. During treatment, the transmit element may be used to determine respiration rate or heart rate, for target tracking (e.g., HIFU can be applied throughout the entire respiration cycle), or to potentially shut down the HIFU treatment should the operator venture too far off the target even with automatic phase delay adjustment. Further, imaging with the intravascular device that tracks the target on the renal artery may be used to communicate this information back to the HIFU treatment device 12 so that energy may still be delivered through the breathing or cardiac cycle. That is, such imaging may track any displacement of the renal artery due to the breathing or cardiac cycle and may accommodate the focus of the HIFU treatment device 12 accordingly. The information may also be used to guide the operator back to the treatment site or to dynamically modify the beam delays or mechanical positioning of HIFU to stay on target.

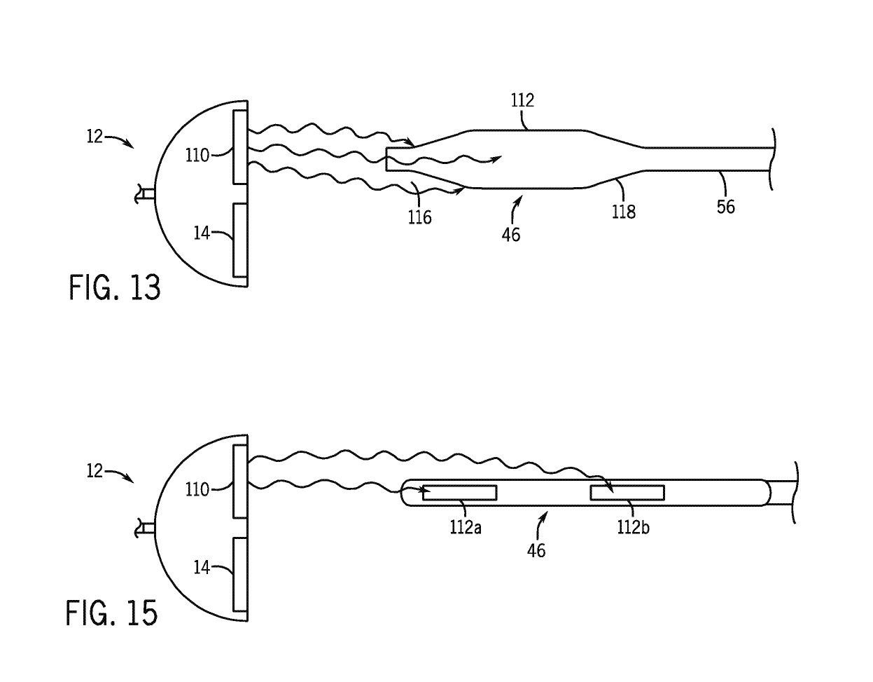

Similar to the acoustic transmitter on the intravascular device that is used to locate the external HIFU treatment device 12 relative to the intended target, the acoustic receiver may also be used to properly place the HIFU device. In certain embodiments, the receiver on the intravascular device can offer a wide acceptance angle due to the geometry. For example, as shown in FIG. 13, a tapered cylindrical receive element 112 with elongated ends 116 and 118 may provide a better acceptance angle than a single square or rectangular element for energy emitted by a transmitter 110. Specially designed devices may be used to improve the acceptance angle (e.g., for receiving) or the directivity (e.g., for transmitting) of the device.

In one embodiment, small elements on an acoustic HIFU transducer 14 can be pulsed serially or coded excitation may be used to help locate the transducer 14 relative to the intended target and guide the physician for proper placement of the device. Arrival times of the sound wave may be used to locate the intended target in three dimensions. If the receiver is a fixed distance away from the intended target, the mechanical offset due to structural design of the intravascular device can potentially be added to the acoustic calculation.

In another technique, the pulse length and amplitude can be optimized. For example, if only one transmit element exists in the transducer 14 (e.g., a focused bowl), then the shortest pulse length can occur at the focal point where all of the sound waves from the bowl constructively interfere at the same time. If the transducer 14 is on the beam axis, yet in the near field or far field, then the amplitude can be lower than expected. If the transducer 14 is off the beam axis, then the amplitude can be lower than expected and the pulse length longer than anticipated. For particular geometries, the peak does not necessarily correspond to the focal point. In this case with the lateral beam symmetry, a two-dimensional presentation may be used to inform the user. One axis can represent depth along the beam axis and the second axis can represent lateral distance from the beam axis. If calculations are difficult to interpret, then a possible LUT may be used for improved calculations to determine the receiver position relative to the HIFU aperture. The receiver (e.g., receiver 90) can have a suitable acceptance angle.

In yet another technique, if the aperture is an annular transducer (e.g., as shown in FIG. 14 in a bottom view of an exemplary patient-contact surface 18 of the housing 16), pulsing of individual rings 120 can be used to center the beam on the receiver. In the depicted embodiment, the annular targeting transmitter 110 is shown as separate from the therapy transducer 14. However, it should be understood that, in any of the examples provided in the present disclosure, the transducer that communicates with the receive element 112 (or a transmitter) on the targeting element 46 may be separate from or combined with the transducer 14. The time of flight of the energy path (e.g., as shown by arrows 122) can be used to determine the focal depth to use for the treatment. Further, if an imaging transducer is coupled to the HIFU treatment device 12, alignment of the transducer 14 to the intended target can be improved by creating an image of the lines received by the element on the intravascular device. In another embodiment, multiple receive elements 112 on the intravascular device 44 may improve the ability to sense the relative lateral direction of the transducer 14 (or dedicated transmitter 110) relative to the intended target. Multiple receive elements, shown as receive elements 112a, 112b in FIG. 15, may also be used to improve overall acceptance angle.

Similarly, in the receive case, the detected intensity of the energy from the HIFU device can be used to calculate attenuation, the required HIFU aperture intensity to achieve the therapeutic effect, and the necessary delays to optimize constructive interference at the target site LUTs. One-way imaging may also help determine if obstructions are preventing delivery of acoustic energy to the target. During treatment, the receive element can be used to track respiration and operator motion, as well as adjust delays, so that therapeutic energy can be effectively delivered to the target.

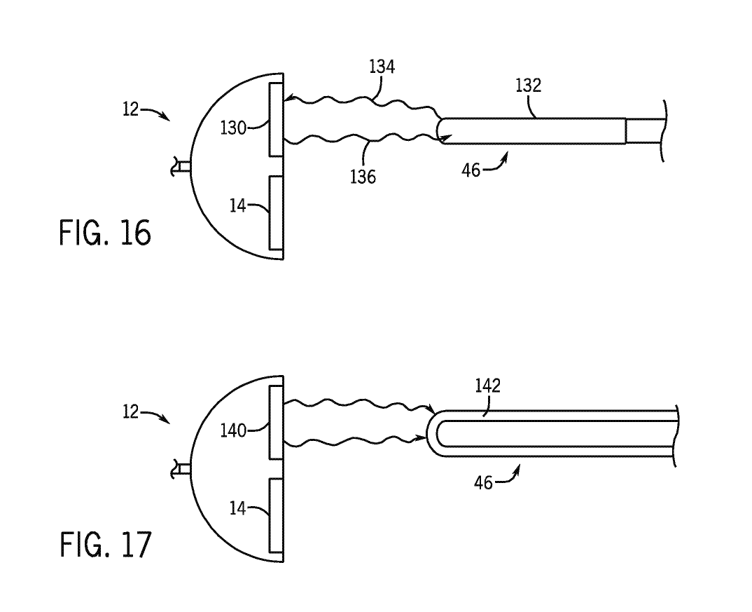

The proposed functionality that can be in the transmitter and the receiver on the intravascular device 44 can be combined (e.g., as illustrated in FIG. 16). In such embodiments, the HIFU treatment device 12 may include a suitable corresponding transmit and receive element (e.g., a transceiver 130). For example, in some embodiments, initial intensity and delay optimization can be accomplished using energy transmitted by the transmit element of a transceiver 132 (e.g., shown as arrow 134), while final tweaking and small adjustments may be performed using the energy received by the receive element of the transceiver 130 (e.g., shown as arrow 136). Having a combined transmitter and receiver can facilitate positioning (e.g., whether the physician needs to move left or right to locate the HIFU treatment device 12 at the intended target). In some embodiments, the transducer and the transceiver may be combined into a single element. Further, in embodiments in which both transceivers 130 and 132 are capable of transmitting and receiving, decisions regarding which element to use for targeting the treatment area may be made on a patient-by-patient basis. For example, the transceiver 130 may be tested. If the signal is weak, the transceiver 132 may be activated.

Another option, depicted in FIG. 17, can be to have a passive material (e.g., material 142) added to the targeting element 46 that can be either highly reflective or absorptive to set the signal apart from other tissue signals that may be backscattered to the HIFU treatment device 12. The material 142 may be a solid, liquid, or gas depending on whether the material is to be highly reflective or absorptive. Furthermore, the material 142 may be shaped or patterned in such a way as to produce a unique signal from sonification by the HIFU transducer that may be detected by a receiver 140. Alternatively, the HIFU treatment device 12 may not employ a dedicated transducer for the targeting element 46 and the transducer 14 may be used. The material 142 may be designed so direction may be determined by the signal intensity or scattering pattern. For example, the material 142 on one side of the intended target may be absorptive, and the material 142 may be reflective on the opposing side. This information can be used to help guide the operator to the intended target. The construction of these intravascular devices may also be used to assist in delivery of enough energy to the target for neuromodulation. For example, a highly reflective material may reduce the overall energy requirement from the HIFU device since the re-radiated field can pass through the focal point and contribute to the heating. This can reduce the energy required from the HIFU and the potential threat to other tissues. Such materials may include thin-layer, high-impedance metals (e.g., aluminum or nitinol). For example, the intravascular devices provided herein may be guided within or proximate to the renal artery. If the distance to the desired treatment area is known, the offset may be used to target the tissue for therapy. The transducer 14 may generate many pulse-echo lines that can be used to help locate the acoustic material and the intended target. The more real-time pulse-echo lines that are created, the higher the likelihood that the material 142 can be located among backscattered tissue signals. The ability to determine where to target may be based, for example, on the beam characteristics from the imaging system and the system of the target. For example, if the beam response from the imaging system is spatially wide, then the ability to spatially determine the location of the target can be reduced.

C. Extracorporeal HIFU Devices with External Targeting Systems

In certain embodiments, the system 10 as provided can be completely external (e.g., extracorporeal), including the treatment device and any associated targeting systems. Such implementations may reduce the procedure cost and improve the patient experience. Use of an external targeting system may facilitate delivery of appropriate energy to the tissue. In certain embodiments, external targeting systems may include a therapy transducer or may be housed within the HIFU treatment device 12. In embodiments in which an additional targeting transducer is employed, the two coordinate systems may be coupled together (e.g., imaging the therapy beam).

1. Ultrasound Transducer External Targeting and Tracking

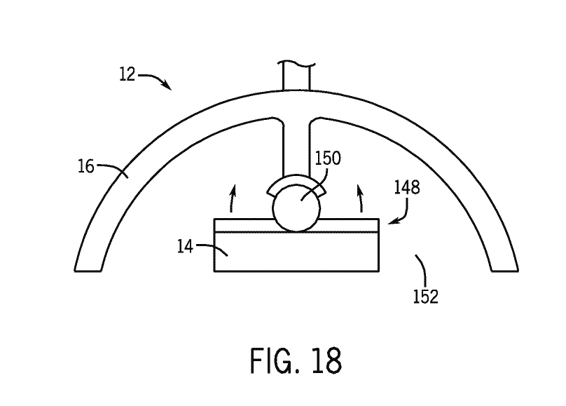

In some embodiments, the transducer 14 may produce B-mode ultrasound images of the kidney and surrounding tissues. The B-mode images may be used in tandem with the location of the therapy beam to target the therapy beam approximately relative to the renal artery. In some embodiments, the therapy beam may be generally perpendicular to the renal wall. In other embodiments, the beam may be targeted approximately parallel or along the renal artery wall to generate a longer lesion. Furthermore, the therapy beam can create a lesion on both the proximal and distal portions of the renal artery. For example, the HIFU treatment device 12 may target multiple foci simultaneously or in a series. The B-mode image may not provide information on how out-of-plane tissues are affected. In order to ensure full treatment of the renal artery, a mechanical or electronic tip-and-tilt system may be added in place of or to supplement manual manipulation devices. An example of a tilt-and-tip transducer mounting 148 is depicted in FIG. 18. The transducer mounting 148 may include a ball-socket mounting 150 that can allow rotational movement within a housing opening 152. The amount of tip and tilt may be determined by depth to the intended target and size of the renal artery (e.g., as determined by the targeting transducer). During the treatment, the targeting transducer may be used to track treatment or actual movement of the HIFU treatment device 12 (e.g., if held manually or if breathing interferers with energy deposition). Such mountings may be used in transducer arrays, and may include micromechanical features.

Given the unique shape and scattering from the kidney, correlation and morphing techniques may be employed to identify the kidney with B-mode images. Identifying the kidney helps localize the renal artery and the possible region of treatment. In addition to identifying the renal artery, the targeting transducer may also ensure that the path to the target is unobstructed. This can be done by using the targeting transducer to “image” the therapy and measure beam symmetry. In other embodiments, the ultrasound information may be used to provide blood-flow or tissue-elasticity information, which, in turn, may be used to distinguish the renal artery from the surrounding tissue. The beam kurtosis and skewness may be used as indicators of possible obstructions, poor coupling, or beam-altering aberrations.

2. 2D B-Mode Images Combined with Doppler

B-mode images may have limitations when trying to distinguish stationary tissue from arterial or venous blood flow. The blood flow can have additional contrast in the B-mode image by using power Doppler or color Doppler. The temporal Doppler signal from the tissue can help to identify arteries from veins. The location of the blood flow can add to the ability to identify artery walls so tissue can be properly targeted. Once the renal artery is located after the arterial tree of the kidney is recognized, the possible treatment sites can be located. Similar to the B-mode images, correlation and morphing techniques along with a LUT may be used to identify the kidney as well as the renal artery that leads to the kidney. In order to treat around the entire circumference of the renal artery, the HIFU may be electronically or mechanically moved. It can be possible to treat both the proximal and distal sides of the renal artery simultaneously relative to the HIFU transducer given therapy-beam geometries. The duplex targeting may be used for motion tracking during actual treatment. In some embodiments, the tissue further from the therapy device can be treated first. As noted, the treatment may also be targeted using just blood flow information, and may not include duplex imaging.

In addition to identifying the renal artery, the therapy and targeting transducers can be used to determine the possible intensity in and around the renal artery by observing the change in the flow pattern due to insonification (i.e., acoustic streaming). This can be measured, for example, during the pulsing of the blood or during the rest time. For example, the HIFU transducer can pulse the renal artery at high intensity for a brief period of time (e.g., causing little or no heating) and the targeting transducer can detect the change in flow. The “direction” and “magnitude” of the flow can suggest the intensity of the acoustic beam within the renal artery. Further, this may help the user/system properly place the focus of the therapy beam to cause the desired tissue effect. In addition to blood-flow information, ultrasound waves may be used to differentiate tissues of different stiffness via elasticity imaging, which can provide a measure of the pliability or stiffness of tissue. Arteries are generally stiff and the artery walls may be located via elasticity imaging. Further, elasticity imaging may be used in conjunction with blood-flow information.

3. Multiplane Imaging

Use of additional imaging planes can increase the confidence that the renal artery is properly targeted. For example, multiple B-mode or Doppler planes may be at an angle to one another or parallel to one another such that a volume can be generated. The multiplane imaging can help in planning the treatment (e.g., such that the entire circumference of the renal artery is addressed and a successful neuromodulation is achieved).

4. Imaging Fusion

Another methodology that can be combined with ultrasound targeting can include MRI or X-ray CT images in combination with real-time ultrasound imaging. In this case, the MRI or CT images can be gathered before the treatment. For example, a combined targeting and therapeutic transducer can be placed on the patient. A computer program can take the ultrasound dataset 2D or 3D and align (i.e., register) the ultrasound image with the image from the MRI or CT dataset using various suitable correlation techniques. The results may be simultaneously displayed on a screen for the physician prior to or during treatment. The ultrasound images or other images can be faded into the background depending on the preference of the physician. After the device is positioned over the target by correlating ultrasound images with the MRI or CT dataset, near-field obstructions can be checked given the a priori knowledge from the MRI or CT dataset combined with ultrasound-beam knowledge. The movement of the beam to treat the entire renal artery can be planned using the high-contrast existing images.

5. Magnetic Tracking after Acquiring Target

Magnetic tracking can be used for an external device after the target is acquired. For example, a magnetic source can sit on a fixed surface (e.g., a bed). The magnetic receiver can sit on the transducer. After the target is acquired, the magnetic tracking system can be used to help the operator stay on target during the therapeutic treatment by using differential analysis. Magnetic tracking can also be used with fusion imaging to further enhance the ability to track motion and movement during treatment. For example, the system may be configured to display the proper imaging plane from the MRI or CT dataset that can be used with the ultrasound image.

6. Stationary Targeting and Therapeutic Belt

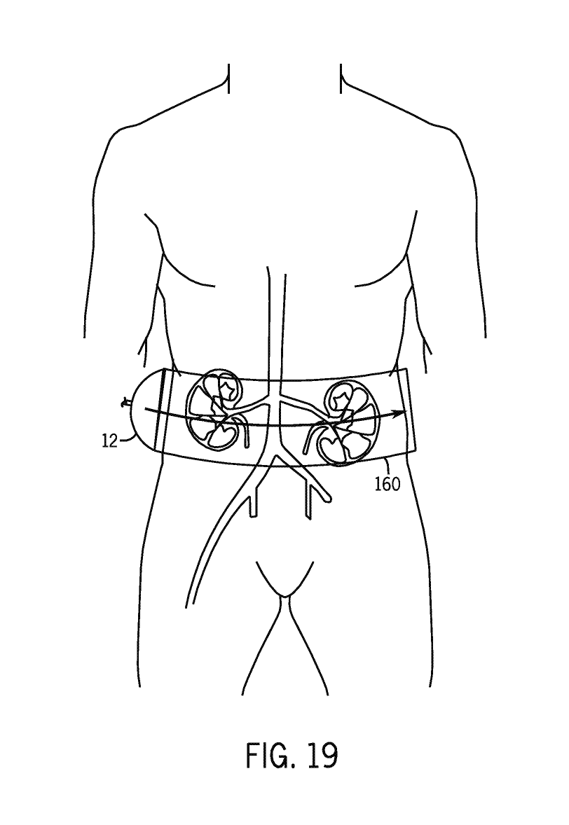

Another technique may include use of a semi-automatic or automatic targeting and treatment device, an example of which is depicted in FIG. 19. One example of such a device may include a belt 160 with an inflatable bladder that can be filled with water. Other embodiments may include a mechanical transducer that can move around the perimeter of the belt 160 for targeting and therapy, a belt 160 populated with targeting and therapy transducers, or a small fluid-filled tub with a coupled transducer. In some embodiments, a device can include a tub connected to an MRI system. The area with the fluid-filled cavity can be configured to address the coupling needs around posterior parts of the abdomen. The imaging and therapy transducers can mechanically slide or move on part of a table. The MRI can still serve to calculate therapy beam acceptability prior to and during treatment.

D. Size and Configuration of the HIFU Focal Points for Achieving Neuromodulation in a Renal Artery

It should be understood that the embodiments provided herein may be used in conjunction with one or more transducers 14. In some patients, it may be desirable to use the transducer(s) 14 to create a single lesion or multiple focal lesions that can be circumferentially spaced along the longitudinal axis of the renal artery. A single focal lesion with desired longitudinal and/or circumferential dimensions, one or more full-circle lesions, multiple circumferentially-spaced focal lesions at a common longitudinal position, and/or multiple longitudinally spaced focal lesions at a common circumferential position alternatively or additionally may be created.

Depending on the size, shape, and number of the transducers 14, the lesions may be circumferentially spaced along the longitudinal axis of the renal artery. In particular embodiments, it can be desirable for each lesion to cover at least about 10% of the vessel circumference to increase the probability of affecting the renal plexus. It also can be desirable that each lesion be positioned into and beyond the adventitia to thereby affect the renal plexus. However, in some embodiments, lesions that are too deep (e.g., greater than 5 mm) run the risk of interfering with non-target tissue and tissue structures (e.g., the renal vein), so a controlled depth of energy treatment also can be desirable.

In certain embodiments, a plurality of focal points of the transducer 14 may be used during treatment. Refocusing the transducer 14 in both the longitudinal and angular dimensions can provide a second treatment site for treating the renal plexus. Energy then may be delivered via the transducer 14 to form a second focal lesion at this second treatment site, thereby creating a second treatment zone. For embodiments in which multiple transducers 14 are associated with a treatment device, an initial treatment may result in two or more lesions, and refocusing may allow additional lesions to be created.

In certain embodiments, the lesions created via refocusing of the transducer 14 can be angularly and longitudinally offset from the initial lesion(s) about the angular and lengthwise dimensions of the renal artery, respectively. Superimposing the lesions created by initial application and repositioning may result in a discontinuous lesion (e.g., the lesion can be formed from multiple, longitudinally and angularly spaced treatment zones). One or more additional focal lesions optionally may be formed via additional refocusing of the transducer 14. In some embodiments, superimposition of all or a portion of the lesions can provide a composite treatment zone that is non-continuous (e.g., that is broken up along the lengthwise dimension or longitudinal axis of the renal artery), yet that is substantially circumferential (i.e., that substantially extends all the way around the circumference of the renal artery over a lengthwise segment of the artery). Furthermore, an automatic system can be configured to store location information for treatments and to shift encoder counts to locate additional treatments relative to previous treatments. Similarly, a registered pre-treatment image can be used to locate one or more treatments.

E. Applying Energy to Tissue Via the Transducer

Referring back to FIG. 5, in the illustrated embodiment, the generator 20 may supply energy to the transducer 14 to generate acoustic waves. Energy delivery may be monitored and controlled, for example, via data collected with one or more sensors, such as temperature sensors (e.g., thermocouples, thermistors, etc.), impedance sensors, pressure sensors, optical sensors, flow sensors, chemical sensors, etc., which may be incorporated into or on the transducer 14 or another suitable intravascular device. A sensor may be incorporated into the transducer 14 in a manner that allows a device to specify whether the sensor is in contact with the skin. Sensor(s) may, for example, be incorporated on the side of the transducer 14 that contacts the skin during power and energy delivery. In embodiments in which intravascular devices are used, particularly those that are located proximate to the renal artery, a sensor may be positioned on or near the tissue at the treatment site and/or facing blood flow. In other embodiments, the ultrasound energy may be delivered in a controlled manner to achieve desired heating of tissue in the range of 60 to 90° C. To prevent overheating and to control temperature, a temperature sensor, such as a thermistor, can be incorporated in the design of the intravascular device. The intravascular device may also be used to assess the lesion(s) during treatment to determine when treatment should stop as well as assess the lesion(s) after treatment. The generator 20 may be equipped with electronic circuits capable of receiving a temperature signal and controlling the energy delivered to the transducer 14. It is appreciated that the temperature control feedback may be incorporated in appropriate designs and embodiments disclosed herein.

Additionally or alternatively, various microsensors may be used to acquire data corresponding to the transducer 14, the vessel wall, and/or the blood flowing across an intravascular device. For example, arrays of micro thermocouples and/or impedance sensors may be implemented to acquire data along the transducer 14 or other parts of the treatment device. Sensor data may be acquired or monitored prior to, simultaneous with, or after the delivery of energy or in between pulses of energy, when applicable. The monitored data may be used in a feedback loop to better control therapy, e.g., to determine whether to continue or stop treatment, and it may facilitate controlled delivery of increased or reduced power or a longer or shorter duration therapy.

F. Cooling the Transducer

Non-target tissue may be protected by blood flow within the respective renal artery that serves as a conductive and/or convective heat sink to carry away excess thermal energy. Further, the monitoring of blood flow pulsatility may be used in conjunction with energy delivery to help reduce the chance of damaging the vessel wall. That is, it may be advantageous to deliver an energy dose during pulsing to reduce damage to the wall. When blood is pulsing through the vessel, the vessel can be less likely to experience stenosis. In particular embodiments, since blood flow is not blocked by the extracorporeal transducer 14, the native circulation of blood in the respective renal artery can serve to remove excess thermal energy from the non-target tissue and the transducer 14. The removal of excess thermal energy by blood flow also can allow for treatments of higher power, where more power may be delivered to the target tissue as heat is carried away from the application site and non-target tissue. In this way, ultrasound energy can heat target neural fibers located proximate to the vessel wall to modulate the target neural fibers, while blood flow within the respective renal artery protects non-target tissue of the vessel wall from excessive or undesirable thermal injury. Since HIFU may employ remote focal points, the highest temperature treatment regions may be located outside or on an exterior surface of a renal artery.

It may also be desirable to provide enhanced cooling by inducing additional native blood flow across the transducer 14. For example, techniques and/or technologies may be implemented by the caregiver to increase perfusion through the renal artery or to the transducer 14. These techniques may include positioning partial-occlusion elements (e.g., balloons) within upstream vascular bodies, such as the aorta, or within a portion of the renal artery to improve flow across the transducer 14.

In addition, or as an alternative, to passively utilizing blood flow as a heat sink, active cooling may be provided to remove excess thermal energy and protect non-target tissues. For example, a thermal fluid infusate may be injected, infused, or otherwise delivered into the vessel in an open-circuit system. Thermal fluid infusates used for active cooling may, for example, include saline (e.g., at room temperature or chilled) or some other biocompatible fluid. The thermal fluid infusate(s) may, for example, be introduced through an intravascular device via one or more infusion lumens and/or ports. When introduced into the bloodstream, the thermal fluid infusate(s) may, for example, be introduced through a guide catheter or at other locations relative to the tissue for which protection is sought. In some embodiments, fluid infusate can be injected through a lumen associated with the intravascular device. The delivery of a thermal fluid infusate in the vicinity of the treatment site (e.g., via an open-circuit system and/or via a closed-circuit system) may, for example, allow for the application of increased/higher power, may allow for the maintenance of lower temperature at the vessel wall during energy delivery, may facilitate the creation of deeper or larger lesions, may facilitate a reduction in treatment time, may allow for the use of a smaller transducer size, or a combination thereof. In some embodiments, in addition to or instead of delivering a thermal fluid infusate, an intravascular cryogenic device can be used to cool tissue at the treatment site. Such a device can be configured to cause cooling, for example, by circulating cooled fluid or by the Joule-Thomson effect alone or in combination with refrigerant phase change.

Accordingly, the system 10 as provided may include an intravascular device with features for an open-circuit cooling system, such as a lumen in fluid communication with a source of infusate and a pumping mechanism (e.g., manual injection or a motorized pump) for injection or infusion of saline or some other biocompatible thermal fluid infusate from outside the patient into the patient's bloodstream during energy delivery. Furthermore, an intravascular device may include one or more ports for injection or infusion of saline or another suitable fluid directly at the treatment site. Such a system may also be used in conjunction with a transducer 14 that can be positioned outside the body.

### IV. ADDITIONAL CLINICAL USES OF THE DISCLOSED APPARATUSES, METHODS, AND SYSTEMS

Although certain embodiments of the present techniques relate to at least partially denervating a kidney to block afferent and/or efferent neural communication, the apparatuses, methods, and systems described herein may also be used for other non-renal treatments. For example, the aforementioned intravascular-device system, or select aspects of such system, may be placed in other peripheral blood vessels to deliver energy and/or electric fields to achieve a neuromodulatory affect by altering nerves proximate to these other peripheral blood vessels. There are a number of arterial vessels arising from the aorta that travel alongside a rich collection of nerves to target organs. Utilizing the arteries to access and modulate these nerves may have clear therapeutic potential in treating a number of disease states. Some examples include the nerves encircling the celiac trunk, the superior mesenteric artery, and the inferior mesenteric artery.

Sympathetic nerves proximate to or encircling the arterial blood vessel known as the celiac trunk may pass through the celiac ganglion and follow branches of the celiac trunk to innervate the stomach, small intestine, abdominal blood vessels, liver, bile ducts, gallbladder, pancreas, adrenal glands, and kidneys. Modulating these nerves either in whole or in part (e.g., via selective modulation) may enable treatment of conditions including, but not limited to, diabetes, pancreatitis, obesity, hypertension, obesity-related hypertension, hepatitis, hepatorenal syndrome, gastric ulcers, gastric motility disorders, irritable bowel syndrome, and autoimmune disorders, such as Crohn's disease.

Sympathetic nerves proximate to or encircling the arterial blood vessel known as the inferior mesenteric artery may pass through the inferior mesenteric ganglion and follow branches of the inferior mesenteric artery to innervate the colon, rectum, bladder, sex organs, and external genitalia. Modulating these nerves either in whole or in part (e.g., via selective modulation) may enable treatment of conditions including, but not limited to, GI motility disorders, colitis, urinary retention, hyperactive bladder, incontinence, infertility, polycystic ovarian syndrome, premature ejaculation, erectile dysfunction, dyspareunia, and vaginismus.

While arterial access and treatments have been provided herein, the disclosed apparatuses, methods, and systems may also be used with suitable veins, lymphatic ducts, or other anatomical structures. Furthermore, partially or entirely extracorporeal treatment systems in accordance with embodiments of the present technology can be well suited for modulation of nerves that are not easily accessible via catheter-based approaches. For example, in some cases, it can be useful to enhance selectivity of treatment with respect to nerves associated with a targeted organ relative to other nerves. In some embodiments, treating nerves closer to a targeted organ can enhance this selectivity. Vessels, however, often decrease in size, branch, and/or become more tortuous as they move closer to organs. Accordingly, intravascular access to nerves near portions of vessels relatively close to organs can be particularly challenging. Partially or entirely extracorporeal treatment systems in accordance with embodiments of the present technology, however, can be configured to treat these nerves with relatively few, if any, constraints based on vascular characteristics.

### V. EXAMPLES

1. A system for extracorporeal renal neuromodulation of a human patient, the system comprising:
a non-invasive ultrasound transducer including a housing configured to be applied to a patient's skin;
an ultrasound energy source operably coupled to the ultrasound transducer,
an extracorporeal targeting system configured to—
   receive information about renal vasculature of the patient; and
   based on the information about the renal vasculature of the patient, focus the ultrasound transducer on one or more focal points in a left renal plexus and/or a right renal plexus of the patient,
wherein the ultrasound transducer is configured to transmit ultrasound energy from the ultrasound energy source to target neural fibers of the left renal plexus and/or the right renal plexus of the patient to thermally induce modulation of the target neural fibers.

2. The system of example 1 wherein the non-invasive ultrasound transducer comprises a first ultrasound transducer, and wherein the extracorporeal targeting system comprises a second ultrasound transducer configured to produce B-mode ultrasound images of kidney and surrounding tissues, and wherein the B-mode images are configured to be used to focus the ultrasound transducer.

3. The system of example 2 wherein the second ultrasound transducer is further configured to provide at least one of blood flow and tissue elasticity information during treatment, and wherein ultrasound energy delivery to the target neural fibers is modified based, at least in part, on feedback from the blood flow and/or tissue elasticity information.

4. The system of example 2 wherein the extracorporeal targeting system further comprises a component configured to deliver power Doppler or color Doppler to the target treatment site, and wherein ultrasound energy delivery to the target neural fibers is modified based, at least in part, on temporal Doppler signals from the treatment site.

5. The system of example 4 wherein the extracorporeal targeting system is configured to utilize multiple B-mode and/or Doppler imaging planes to receive information about the renal vasculature of the patient.

6. The system of example 1 wherein the extracorporeal targeting system is configured to generate MRI or X-ray CT images before therapy, and further wherein the extracorporeal targeting system is configured to focus the ultrasound transducer on one or more focal points using the MRI or X-ray CT images in conjunction with real-time ultrasound imaging of the renal vasculature.

7. The system of example 1 wherein the extracorporeal targeting system comprises a magnetic tracking system including a magnetic source coupled to a fixed surface proximate to the patient and a magnetic receiver carried by the ultrasound transducer.

8. The system of example 1 wherein the extracorporeal targeting system comprises a belt including an inflatable bladder and configured to be attached to the skin of the patient, and wherein the belt is populated with a plurality of targeting transducers.

9. The system of any one of examples 1 to 8 wherein the extracorporeal targeting system is configured to focus ultrasound energy at target neural fibers approximately one to three millimeters away from a wall of a renal artery of the patient.

10. The system of any one of examples 1 to 9 wherein ultrasound energy delivery via the ultrasound transducer is timed to at least generally correspond to one or more body cycles of the patient.

11. The system of example 10 wherein ultrasound energy delivery via the ultrasound transducer is timed to at least generally correspond to respiration of the patient.

12. The system of example 10 wherein ultrasound energy delivery via the ultrasound transducer is timed to at least generally correspond to heart rate of the patient.

13. The system of any one of examples 1 to 12 wherein the one or more focal points comprise at least two focal points spaced apart about less than 5 mm in the left renal plexus and/or the right renal plexus of the patient.

14. A method for renal neuromodulation of a human patient, the method comprising:
locating one or more focal points comprising target neural fibers of a left renal plexus and/or a right renal plexus of the patient via a targeting system external to the patient,
delivering ultrasound energy from an extracorporeal ultrasound transducer to the one or more focal points; and
thermally inhibiting neural activity along the neural fibers via the ultrasound energy from the extracorporeal ultrasound transducer.

15. The method of example 14 wherein the extracorporeal ultrasound transducer is positioned on the patient's skin.

16. The method of any one of examples 14 to 15 wherein the extracorporeal ultrasound transducer comprises an array of transducers, and wherein delivering ultrasound energy comprises focusing the energy of the array of transducers onto one or more overlapping focal points.

17. The method of any one of examples 14 to 16, further comprising emitting nontherapeutic ultrasound energy from the extracorporeal ultrasound transducer and determining a position or geometry of a renal artery of the patient based on the nontherapeutic ultrasound energy.

18. The method of example 17 wherein determining a position or geometry of the renal artery based on the nontherapeutic ultrasound energy comprises measuring symmetry, kurtosis, or skewness of the nontherapeutic ultrasound energy.

19. The method of example 17 wherein determining a position or geometry of the renal artery based on the nontherapeutic ultrasound energy comprises using Doppler imaging in conjunction with the nontherapeutic ultrasound energy.

20. A system for extracorporeal modulation of renal nerves, the system comprising:
an ultrasound transducer having a housing configured to be applied to a patient's skin;
a catheter comprising a proximal portion and a distal portion, the distal portion configured for intravascular delivery proximate to a renal artery of the patient, wherein the distal portion of the catheter comprises a device configured to provide information about a geometry or location of the renal artery; and
a monitoring device configured to—
   receive the information about the renal artery; and
   focus the ultrasound transducer based on the information about the renal artery on one or more focal points on a wall of the renal artery to modulate renal nerves.

21. The system of example 20 wherein the catheter is configured for intravascular delivery via a 6 French or smaller guide catheter.

22. The system of any one of examples 20 to 21 wherein the device configured to provide information about the geometry or location of the renal artery comprises an imaging ultrasound transducer.

23. The system of any one of examples 20 to 22 wherein the device configured to provide information about the geometry or location of the renal artery comprises an acoustical transmitter.

24. The system of example 23 wherein the ultrasound transducer is configured to receive signals from the acoustical transmitter.

25. The system of example 23, further comprising an acoustical receiver associated with the housing and configured to receive signals from the acoustical transmitter.

26. The system of example 20 wherein the device configured to provide information about the geometry or location of the renal artery comprises an acoustical receiver.

27. The system of example 26, further comprising an acoustical transmitter associated with the housing and configured to transmit signals to the acoustical receiver.

28. The system of example 20 wherein the device configured to provide information about the geometry or location of the renal artery comprises a magnetic receiver or a magnetic transmitter.

29. The system of example 28, further comprising a magnetic transmitter associated with the housing and configured to transmit signals to the magnetic receiver.

30. The system of example 28, further comprising a second magnetic receiver associated with the housing and an external transmitter configured to transmit signals to the first and second magnetic transmitters.

31. The system of example 28, further comprising a magnetic receiver associated with the housing and configured to receive signals from the magnetic transmitter.

32 The system of example 28, further comprising an ultrasound transducer associated with an intravascular device and configured to provide imaging information.

33. The system of any one of examples 20 to 32 wherein the distal portion of the catheter comprises an acoustic reflector or absorber.

34. The system of any one of examples 20 to 33 wherein the ultrasound transducer comprises an annular transducer or an array of transducers.

35. The system of any one of examples 20 to 34 wherein the one or more focal points comprise at least two focal points spaced apart about less than 5 mm.

36. A method of renal neuromodulation, the method comprising:
locating one or more focal points at renal nerves positioned on or outside a renal artery of a human patient via an intravascular targeting system;
emitting ultrasound energy from an extracorporeal ultrasound transducer;
focusing the ultrasound energy on the one or more focal points; and
heating the renal nerves at the focal points with the focused ultrasound energy.

37. The method of example 36 wherein the extracorporeal ultrasound transducer is positioned on the patient's skin.

38. The method of any one of examples 36 to 37, further comprising emitting ultrasound energy from an imaging ultrasound transducer positioned within or proximate the renal artery.

39. The method of any one of examples 36 to 38 wherein focusing the ultrasound energy comprises using imaging information from the imaging ultrasound transducer.

40. The method of any one of examples 36 to 39 wherein the extracorporeal ultrasound transducer comprises an array of transducers, and wherein focusing the ultrasound energy comprises focusing the energy of the array of transducers onto one or more overlapping focal points.

41. The method of any one of examples 36 to 40, further comprising emitting nontherapeutic ultrasound energy from the extracorporeal ultrasound transducer and determining a position or geometry of the renal artery based on the nontherapeutic ultrasound energy.

42. The method of example 41 wherein determining a position or geometry of the renal artery based on the nontherapeutic ultrasound energy comprises measuring symmetry, kurtosis, or skewness of the nontherapeutic ultrasound energy.

43. The method of example 41 wherein determining a position or geometry of the renal artery based on the nontherapeutic ultrasound energy employs Doppler imaging.

44. A treatment system for extracorporeal modulation of renal nerves of a human patient, the system comprising:
a therapy device comprising—
   a therapy ultrasound transducer associated with a housing and configured to transmit therapy energy through skin of the patient to modulate the renal nerves;
   a targeting transducer associated with the housing and configured to transmit targeting energy through the skin, wherein the targeting energy is not sufficient to modulate the renal nerves; and
   a focusing structure configured to focus the therapy energy emitted by the therapy ultrasound transducer at a focal point located on or outside a renal artery of the patient; and
a monitor coupled to the therapy device, the monitor comprising—
   an input circuit configured to receive a signal from the targeting transducer,
   a memory storing an algorithm configured to determine one or more characteristics of the renal artery based on the signal from the targeting transducer and to provide instructions to the focusing structure based on the one or more characteristics; and
   a processor configured to execute the algorithm.

45. The system of example 44 wherein the algorithm is configured to determine a geometry or location of the renal artery based on the signal from the targeting transducer.

46. The system of any one of examples 44 to 45 wherein the algorithm is configured to determine the location of the renal artery relative to a kidney.

47. The system of any one of examples 44 to 46 wherein the algorithm is configured to determine one or more characteristics of the renal artery based on the signal from the therapy transducer.

48. The system of any one of examples 44 to 47 wherein the therapy transducer or the targeting transducer is configured to actuate relative to the housing.

### VI. CONCLUSION

The above detailed descriptions of embodiments of the disclosure are not intended to be exhaustive or to limit the disclosure to the precise forms disclosed above. Although specific embodiments of, and examples for, the disclosure are described above for illustrative purposes, various equivalent modifications are possible within the scope of the disclosure, as those skilled in the art will recognize. For example, while steps are presented in a given order, alternative embodiments may perform steps in a different order. The various embodiments described herein may also be combined to provide further embodiments.

From the foregoing, it will be appreciated that specific embodiments of the disclosure have been described herein for purposes of illustration, but well-known structures and functions have not been shown or described in detail to avoid unnecessarily obscuring the description of the embodiments of the disclosure. Where the context permits, singular or plural terms may also include the plural or singular term, respectively. For example, much of the disclosure herein describes a transducer 14 in the singular. It should be understood that this disclosure does not exclude two or more transducers.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” and the like are used throughout this disclosure to mean including at least the recited feature(s) such that any greater number of the same feature and/or additional types of other features are not precluded. It will also be appreciated that specific embodiments have been described herein for purposes of illustration, but that various modifications may be made without deviating from the disclosure. Accordingly, the disclosure is not limited except as by the appended claims.

1. A system for extracorporeal renal neuromodulation of a human patient, the system comprising:
a non-invasive ultrasound transducer including a housing configured to be applied to a patient's skin;
an ultrasound energy source operably coupled to the ultrasound transducer;
an extracorporeal targeting system configured to—
receive information about renal vasculature of the patient; and
based on the information about the renal vasculature of the patient, focus the ultrasound transducer on one or more focal points in a left renal plexus and/or a right renal plexus of the patient,
wherein the ultrasound transducer is configured to transmit ultrasound energy from the ultrasound energy source to target neural fibers of the left renal plexus and/or the right renal plexus of the patient to thermally induce modulation of the target neural fibers.

2. The system of claim 1 wherein the non-invasive ultrasound transducer comprises a first ultrasound transducer, and wherein the extracorporeal targeting system comprises a second ultrasound transducer configured to produce B-mode ultrasound images of kidney and surrounding tissues, and wherein the B-mode images are configured to be used to focus the ultrasound transducer.

3. The system of claim 2 wherein the second ultrasound transducer is further configured to provide at least one of blood flow and tissue elasticity information during treatment, and wherein ultrasound energy delivery to the target neural fibers is modified based, at least in part, on feedback from the blood flow and/or tissue elasticity information.

4. The system of claim 2 wherein the extracorporeal targeting system further comprises a component configured to deliver power Doppler or color Doppler to the target treatment site, and wherein ultrasound energy delivery to the target neural fibers is modified based, at least in part, on temporal Doppler signals from the treatment site.

5. The system of claim 4 wherein the extracorporeal targeting system is configured to utilize multiple B-mode and/or Doppler imaging planes to receive information about the renal vasculature of the patient.

6. The system of claim 1 wherein the extracorporeal targeting system is configured to generate MRI or X-ray CT images before therapy, and further wherein the extracorporeal targeting system is configured to focus the ultrasound transducer on one or more focal points using the MRI or X-ray CT images in conjunction with real-time ultrasound imaging of the renal vasculature.

7. The system of claim 1 wherein the extracorporeal targeting system comprises a magnetic tracking system including a magnetic source coupled to a fixed surface proximate to the patient and a magnetic receiver carried by the ultrasound transducer.

8. The system of claim 1 wherein the extracorporeal targeting system comprises a belt including an inflatable bladder and configured to be attached to the skin of the patient, and wherein the belt is populated with a plurality of targeting transducers.

9. The system of claim 1 wherein the extracorporeal targeting system is configured to focus ultrasound energy at target neural fibers approximately one to three millimeters away from a wall of a renal artery of the patient.

10. The system of claim 1 wherein ultrasound energy delivery via the ultrasound transducer is timed to at least generally correspond to one or more body cycles of the patient.

11. The system of claim 10 wherein ultrasound energy delivery via the ultrasound transducer is timed to at least generally correspond to respiration of the patient.

12. The system of claim 10 wherein ultrasound energy delivery via the ultrasound transducer is timed to at least generally correspond to heart rate of the patient.

13. The system of claim 1 wherein the one or more focal points comprise at least two focal points spaced apart about less than 5 mm in the left renal plexus and/or the right renal plexus of the patient.

14. A method for renal neuromodulation of a human patient, the method comprising:
locating one or more focal points comprising target neural fibers of a left renal plexus and/or a right renal plexus of the patient via a targeting system external to the patient;
delivering ultrasound energy from an extracorporeal ultrasound transducer to the one or more focal points; and
thermally inhibiting neural activity along the neural fibers via the ultrasound energy from the extracorporeal ultrasound transducer.

15. The method of claim 14 wherein the extracorporeal ultrasound transducer is positioned on the patient's skin.

16. The method of claim 14 wherein the extracorporeal ultrasound transducer comprises an array of transducers, and wherein delivering ultrasound energy comprises focusing the energy of the array of transducers onto one or more overlapping focal points.

17. The method of claim 14, further comprising emitting nontherapeutic ultrasound energy from the extracorporeal ultrasound transducer and determining a position or geometry of a renal artery of the patient based on the nontherapeutic ultrasound energy.

18. The method of claim 17 wherein determining a position or geometry of the renal artery based on the nontherapeutic ultrasound energy comprises measuring symmetry, kurtosis, or skewness of the nontherapeutic ultrasound energy.

19. The method of claim 17 wherein determining a position or geometry of the renal artery based on the nontherapeutic ultrasound energy comprises using Doppler imaging in conjunction with the nontherapeutic ultrasound energy.

20. A system for extracorporeal modulation of renal nerves, the system comprising:
an ultrasound transducer having a housing configured to be applied to a patient's skin;
a catheter comprising a proximal portion and a distal portion, the distal portion configured for intravascular delivery proximate to a renal artery of the patient, wherein the distal portion of the catheter comprises a device configured to provide information about a geometry or location of the renal artery; and
a monitoring device configured to—
receive the information about the renal artery; and
focus the ultrasound transducer based on the information about the renal artery on one or more focal points on a wall of the renal artery to modulate renal nerves.

21-48. (canceled)
